(19) United States
(12) Patent Application Publication
(10) Pub. No.: US 2011/0014185 A1
(43) Pub. Date: Jan. 20, 2011

(54) METHODS INVOLVING GRAF POLYPEPTIDES

(76) Inventors: Gary J. Doherty, Cambridge (GB); Richard Lundmark, Cambridge (GB); Harvey T. McMahon, Cambridge (GB)

Correspondence Address: GIFFORD, KRASS, SPRINKLE,ANDERSON & CITKOWSKI, P.C, PO BOX 7021, TROY, MI, 48007-7021, US

(21) Appl. No.: 12/919,738

(22) PCT Filed: Feb. 26, 2009
(86) PCT No.: PCT/GB2009/000535

§ 371 Date: Aug. 26, 2010

(30) Foreign Application Priority Data
Feb. 26, 2008 (GB) 0803464.7

(51) Int. Cl. A61K 39/395; G01N 33/566; C12Q 1/34; G01N 33/567; C07K 14/435; A61K 38/02; A61P 35/00; A61P 37/06; A61K 31/713; C07K 16/00; C07K 2/00; C07H 21/02

(52) U.S. Cl. 424/130.1; 435/7.1; 435/18; 435/7.21; 530/350; 514/19.3; 514/44 A; 530/387.1; 530/300; 536/24.5

(57) ABSTRACT

The invention relates to a method of identifying a modulator of clathrin-independent endocytosis, said method comprising
(i) providing a GRAF protein, said GRAF protein comprising a GAP domain;
(ii) providing a candidate modulator; and
(iii) determining the effect of said candidate modulator on the GAP activity of said GRAF protein,
   wherein a change in GAP activity of said GRAF protein in the presence of said candidate modulator identifies said candidate modulator as a modulator of clathrin-independent endocytosis. The invention also relates to GRAF proteins and to methods of manufacture of GRAF modulators.

### BACKGROUND TO THE INVENTION

With the advent of the electron microscope came an appreciation of the enormous complexity of human cellular anatomy. Importantly, this allowed researchers to visualise the network of membranes that exist in each cell. Each human cell has both a large number of internal membranes, in addition to an outer membrane known as the plasma membrane. This plasma membrane defines the limits of the cell in space and functions as a barrier between the interior of the cell and its external environment. It is at the plasma membrane that the cell interacts with its environment. These myriad interactions are mediated by proteins either associated with, or spanning, the lipids that make up this membrane. In order to control these vital interactions, both the protein and lipid constituents of the plasma membrane must be precisely regulated. Endocytosis is the process whereby entirely internal membranes are made de novo from regions of the plasma membrane. In endocytosis, lipids and lipid-associated proteins become fully internalised into the cell. Endocytosis can happen within seconds or minutes, and can therefore swiftly change the composition of the plasma membrane. In order for endocytosis to take place, the plasma membrane must first be indented at the appropriate site: this indentation is known as a plasma membrane invagination. While the plasma membrane is a relatively flat structure, the invaginated membranes are highly curved. An endocytic invagination must be actively generated and maintained by cellular proteins. For several decades, research into endocytosis has focussed primarily on a single endocytic pathway mediated by a protein called Clathrin (Clathrin-mediated endocytosis). Clathrin forms a basket-like lattice around the internalising membranes of this pathway. Much is known about how Clathrin, and a multitude of other proteins involved in Clathrin-mediated endocytosis, produce functional endocytic events. Clathrin-mediated endocytosis is certainly an important endocytic mechanism, but it has recently become clear that other endocytic mechanisms exist that are both independent of Clathrin and morphologically distinct. With few important exceptions, research into these modes of endocytosis has been severely hampered by a lack of knowledge about proteins that specifically mark the internalising membranes and that are necessary for these pathways to function. While some proteins have been heavily implicated in these processes, the mechanisms of Clathrin-independent endocytosis are unclear, which is a problem in the art.

Known approaches to inhibiting cellular migration and cancer cell invasion are rather unspecific and interfere with a large number of pathways.

Known techniques have no way of inhibiting the clathrin-independent endocytic pathway that does not rely on protein depletion or on destructive techniques such as microtubule depolymerisation. This is a problem in the art.

The invention embraces numerous medical indications, including for the treatment of invasive solid organ malignancies, and for specific immunosuppression. Known treatments for each of these are currently manifold, but most rely on the inhibition of proliferation of rapidly dividing cells with a great deal of side effects and are rarely curative, which is a significant drawback in the field.

Clathrin-independent endocytosis, the cytoskeleton, the turnover of adhesion sites, and cellular migration are each currently confusing areas. These fields are intricately linked and interdependent. These issues create problems in the study and dissection of molecular events in these areas.

The present invention seeks to overcome problem(s) associated with the prior art.

### SUMMARY OF THE INVENTION

The present inventors disclose herein for the first time the connection between GRAF protein and clathrin-independent endocytosis, and the myriad biological events which rely on or are controlled by this process, such as cell migration. Furthermore, the inventors disclose specific, biological roles for GRAF protein (such as GRAF1 protein) in co-ordination of these vital cellular processes. In addition, the inventors go on to disclose the remarkable finding that the GRAF GAP domain is central to co-ordination of these events. The invention is based upon these findings.

Thus in one aspect the invention provides a method of identifying a modulator of clathrin-independent endocytosis, said method comprising providing a GRAF protein, said GRAF protein comprising a GAP domain; providing a candidate modulator and determining the effect of said candidate modulator on the GAP activity of said GRAF protein, wherein a change in GAP activity of said GRAF protein in the presence of said candidate modulator identifies said candidate modulator as a modulator of clathrin-independent endocytosis.

In another aspect, the invention relates to a method as described above, said method comprising providing first and second samples of a GRAF protein, said GRAF protein comprising a GAP domain; providing a candidate modulator; contacting said second sample of GRAF protein with said candidate modulator; determining the effect of said candidate modulator on the GAP activity of said GRAF protein by assaying the GAP activity of said first and second samples of GRAF protein; wherein a difference in GAP activity between said first and second samples of GRAF protein identifies said candidate modulator as a modulator of clathrin-independent endocytosis.

The first said sample may be referred to as a reference sample. It may be that in some embodiments of the invention, the reference sample and the test sample or samples may not be processed at the same time. For example, the reference values may be determined and stored and then further test values compared to those stored reference values in order to save the labour of repeating the reference sample analysis in each iteration of the method. Clearly, tests or methods according to the present invention are most scientifically robust when the samples are processed in parallel, under the same conditions, at the same time. Thus, in a preferred embodiment, at least one reference sample is processed for comparison purposes at each iteration of the method of the invention.

In another aspect, the invention relates to a method as described above wherein when said GAP activity is higher in said second sample than said first sample, the candidate modulator is identified as a stimulator or promoter of clathrin-independent endocytosis.

In another aspect, the invention relates to a method as described above wherein when said GAP activity is lower in said second sample than said first sample, the candidate modulator is identified as an inhibitor or suppressor of clathrin-independent endocytosis.

Suitably said GAP activity is assayed using RhoA as a substrate GTPase. Suitable methods for assaying the GAP activity are discussed below.

Suitably said GRAF protein comprises a polypeptide of at least 200 amino acid residues, and wherein said polypeptide comprises a GRAF GAP domain having at least 60% identity to the amino acid sequence 364-563 of human GRAF1. Suitably said polypeptide comprises amino acid sequence corresponding to at least amino acids 364-563 of human GRAF1.

In another aspect, the invention relates to a method as described above further comprising performing an endocytic assay.

In another aspect, the invention relates to a method as described above further comprising performing an adhesion assay.

In another aspect, the invention relates to a method as described above further comprising performing a selectivity assay.

In another aspect, the invention relates to a method as described above further comprising assaying for modulators of FAK activity in vitro.

In another aspect, the invention relates to a method as described above further comprising assaying for modulators of GRAF RhoGAP activity in vitro.

In another aspect, the invention relates to a method as described above further comprising assaying for modulators of GRAF-FAK interaction in vitro.

In another aspect, the invention relates to a method as described above further comprising assaying for changes in GRAF distribution in cells.

In another aspect, the invention relates to a method as described above further comprising assaying for specific modulators of endocytic routes in vivo.

In another aspect, the invention relates to a method as described above further comprising comparison to the GAP activity of a third sample of GRAF1 protein, said third sample comprising a GRAF protein harbouring a mutation in its GAP domain corresponding to the R412D mutation of human GRAF1 .

In another aspect, the invention relates to a method as described above further comprising the step of manufacturing a quantity of the identified modulator of clathrin-independent endocytosis.

In another aspect, the invention relates to use of a modulator of clathrin-independent endocytosis identified according to a method as described above in the manufacture of a medicament for immunosuppression.

In another aspect, the invention relates to use of a modulator of clathrin-independent endocytosis identified according to a method as described above in the manufacture of a medicament for cancer, wherein said cancer is a solid cell malignancy.

In another aspect, the invention relates to a GRAF polypeptide comprising a mutation at the amino acid residue corresponding to amino acid 412 of human GRAF1. Suitably said R412 mutation is R412D. Suitably said GRAF polypeptide comprises the amino acid sequence of human GRAF1 together with the R412D mutation.

### DETAILED DESCRIPTION OF THE INVENTION

GRAF1 regulates a major clathrin-independent endocytic pathway responsible for internalisation of factors including bacterial endotoxins, GPI-linked proteins, and extracellular liquid, and has a central regulatory role in cell migration.

A large volume of membrane redistribution is necessary for the morphological changes occurring in cells undergoing migration, differentiation, cytokinesis, and dendritic arborisation. GTPase Regulator Associated with Focal Adhesion Kinase-1 (GRAF1) is a brain-enriched member of the Oligophrenin family of proteins. Oligophrenin is necessary for normal dendritic spine morphology and mutations in this protein lead to X-linked, non-syndromic mental retardation. Here we show that a major interacting protein of GRAF1 is dynamin, and that N-terminal BAR and PH domains of GRAF1 regulate the formation of dynamic, microtubule- and RhoA-dependent membrane tubules which are endocytic in nature and which are clathrin-independent. GRAF1 also forms complexes with proteins implicated in focal adhesion turnover. Furthermore, many GRAF1-positive tubules emanate from focal adhesions and GRAF1 plays a role in focal adhesion disassembly. GRAF1-positive tubules are prevalent in fibroblasts and GRAF1-mediated trafficking is essential for the endocytic trafficking of Shiga Toxin to the Golgi, for the bulk phase uptake of exogenous substances and trafficking of membranes, and for normal cellular morphology maintenance and migration. These results help us to understand the molecular basis of the neuronal phenotypes associated with mutations in Oligophrenin, as well as characterising a pathway which appears to be as important as the canonical clathrin-dependent pathway in fibroblast endocytosis.

We show that the GAP domain-containing protein GRAF1 is involved in the endocytosis of adhesion receptors and couples this endocytosis to the coordination of cellular migration in concert with Focal Adhesion Kinase. GRAF1 acts as a classical tumour suppressor in haematopoietic cells, where it likely leads to the increased plasma membrane concentration of prosurvival/proproliferation receptors. In solid organ cancers however, GRAF1 and FAK are upregulated and we have shown that this protein is essential for cellular migration, which occurs during the process of tumour cell invasion and metastasis. Similar migratory processes occur in effector cells of the immune system.

We have shown how the function of GRAFT can be inhibited (for example by siRNA treatment) and activated (for example by inhibition of a Rho effector kinase). We address provision of a specific inhibitor of this pathway so that we can study, for example in animal models, its effectiveness in inhibiting cellular migration. Such a drug, has utility as an anti-invasive agent and may be used to treat invading cancers as well as being a potent immunosuppressive agent.

In contrast to prior art techniques, our GRAF protein target is very specifically involved with one process and it is reasonable to expect from our evidence that specific inhibition of our target will have a higher therapeutic index than currently known therapies.

Although in general the techniques mentioned herein are well known in the art, reference may be made in particular to Sambrook et al., Molecular Cloning, A Laboratory Manual (1989) and Ausubel et al., Short Protocols in Molecular Biology (1999) 4th Ed, John Wiley & Sons, Inc.

### GRAF Protein

The terms ‘GRAF Protein’ and ‘GRAF polypeptide’ are used interchangeably herein. The terms are used to refer to polypeptide(s) which are members of the GRAF family, suitably members of the GRAF 1 family.

Members of the GRAF family, or the GRAF 1 family, are well defined. For example, proteins belonging to this family are typically defined with reference to sequence homology (sequence identity) and the presence of particular domains or motifs within the protein which are in common with other GRAF family members.

FIG. 4 and FIG. 5 show phylogenetic trees (one with organisms and one with accession numbers) of the 4 GRAF paralogues. It should be noted that the invention includes each of these. The phylogenetic trees, particularly the tree of accession numbers—may be interpreted as contributing definition of GRAF proteins (e.g. including GRAF-like proteins)—In some embodiments these may represent a definition of GRAF protein according to the present invention. It is an advantage of the invention that these GRAF proteins all function in similar ways in different cell types. These GRAF proteins are discussed in more detail in the examples section.

Suitably, a GRAF protein comprises a protein derived from or related to GRAF 1. Examples of GRAF 1 family proteins include GRAF 1, GRAF 2, OPHN 1 and GRAF 3. Suitably the GRAF protein of the invention is a BAR domain containing protein, more suitably the GRAF 1 protein of the invention is a GRAF 1 family member: GRAF 1 family members and their specific properties are discussed in more detail below.

GRAF proteins according to the present invention may be selected from the group consisting of GRAFT GAP; GRAF2 GAP; GRAF3 GAP; OPHN1 GAP; P50 GAP; P190 GAP and AbrGAP. GRAF proteins according to the present invention may alternatively comprise the sequence of one or more such proteins.

The GRAF protein, such as GRAF 1 protein, of the invention should be of a sufficient size to exhibit its biochemical function of interest. Typically, this means that the GRAF 1 protein must be large enough to comprise a functional GAP domain (if the GAP domain so comprised is indeed functional). In other words, suitably the GRAF protein of the invention comprises an amino acid sequence corresponding to at least the GAP domain of a GRAF family member, such as a GRAF 1 family member. GAP domains are easily identified by the person skilled in the art, in particular with reference to the guidance provided herein. Nevertheless, should any further guidance be required, it should be noted that the gap domain of GRAF 1 is found at amino acid residues 364 to 563 of GRAF 1 (human GRAF1).

Suitably the GRAF protein of the invention is a mammalian GRAF protein. More suitably, a GRAF protein of the invention is a human GRAF protein, or possesses the required characteristics with reference to human GRAF protein, suitably human GRAF 1. In this, regard, suitably the GRAF protein of the invention comprises, comprises amino acid sequence from, or is defined in relation to or derived from, human GRAF 1. In other words, human GRAF1 is the preferred reference sequence for GRAF proteins described herein. More specifically, suitably the reference sequence for human GRAF 1 is the amino acid sequence as defined by NP_{—}055886. For the avoidance of doubt the sequence disclosed in that accession number is incorporated herein by reference. Furthermore, for the avoidance of doubt, the sequence comprised by this human GRAF 1 accession number is specifically incorporated herein by reference in its form at the filing date of this application. Moreover, reference is made to the accompanying figures of this application, which present GRAF protein sequences for ease of reference.

In case further guidance is needed, the preferred GRAF1 sequence is as follows:

GRAF1 full length sequence—814 amino acids—accession number NP_{—}055886; more preferably GRAF1 full sequence corresponds to the splice variant which results in a 759 amino acid sequence—the amino acids different between the two (the 814aa and the 759aa variants) are underlined:

| | |
|---|---|
| 1 | mglpalefsd ccldsphfre tlksheaeld ktnkfikeli kdgkslisal knlssakrkf |
| 61 | adslnefkfq cigdaetdde mciarslqef atvlrnlede rirmienase vlitplekfr |
| 121 | keqigaakea kkkydketek ycgilekhln lsskkkesql qeadsqvdlv rqhfyevsle |
| 181 | yvfkvqevqe rkmfefvepl laflqglftf yhhgyelakd fgdfktqlti siqntrnrfe |
| 241 | gtrseveslm kkmkenpleh ktispytmeg ylyvqekrhf gtswvkhyct yqrdskqitm |
| 301 | vpfdqksggk ggedesvilk sctrrktdsi ekrfcfdvea vdrpgvitmq alseedrrlw |
| 361 | meamdgrepv ynsnkdsqse gtaqldsigf siirkcihav etrgineqgl yrivgvnsrv |
| 421 | qkllsvlmdp ktasetetdi caeweiktit salktylrml pgplmmyqfq rsfikaakle |
| 481 | nqesrvseih slvhrlpekn rqmlqllmnh lanvannhkq nlmtvanlgv vfgptllrpq |
| 541 | eetvaaimdi kfqnivieil ienhekifnt vpdmpltnaq lhlsrkkssd skppscserp |
| 601 | ltlfhtvqst ekqeqrnsii nsslesvssn pnsilnssss lqpnmnssdp dlavvkptrp |
| 661 | nslppnpspt splspswpmf sapsspmpts stssdsspvr svagfvwfsv aavvlslars |
| 721 | slhavfsllv nfvpchpnlh llfdrpeeav hedsstpfrk akalyackae hdselsftag |
| 781 | tvfdnvhpsq epgwlegtln gktglipeny vefl |

In another embodiment the GRAF reference sequence may be a GRAF consensus sequence presented herein. Suitably the GRAF reference sequence is the human GRAF1 sequence discussed above.

Suitably the GRAF protein is or is derived from a human GRAF sequence as explained above. Specifically, the GRAF protein of the invention suitably comprises at least about 200 amino acid residues, suitably at least 250 residues, suitably at least 300 residues, suitably at least 400 residues, suitably at least 500 residues, suitably at least substantially all of the residues of a human GRAF 1 polypeptide.

Suitably the GRAF protein of the invention possesses at least 50% sequence identity to the amino acid sequence of human GRAF 1, suitably at least 60% identity, suitably at least 70% identity, suitably at least 80% identity, suitably at least 90% identity, suitably at least 95% identity, suitably at least 98% identity, suitably at least 99% identity or most suitably the GRAF protein of the invention corresponds to the full amino acid sequence of human GRAF 1 polypeptide.

Where less than the entire sequence of a human GRAF 1 polypeptide is used as the GRAF protein according to the invention, suitably a sequence at least corresponding to the GAP domain of GRAF is used. The above remarks in connection with sizes and/or sequence identity of the GRAF protein of the invention should be interpreted with this in mind. In more detail, if only 200 amino acid residues are present in the GRAF protein of the invention, suitably those correspond to the 200 amino acid residues of the GAP domain of GRAF. For ease of reference, the GAP domain of GRAF is the amino acid sequence from 364 to 563 of human GRAF 1.

In some embodiments the sequence identity may be judged across the GAP domain. In other embodiments the sequence identity may be judged across the whole length of the reference sequence. In other embodiments the sequence identity may be judged across the whole length of the target sequence such as the GRAF protein sequence of the invention.

In general, it is desirable to use as much as possible of the protein of interest so that the domains of interest are present in the context of their naturally occurring amino acid neighbours. Thus, suitably longer GRAF polypeptides are used, most suitably full length GRAF polypeptides are used.

Suitably the GRAF1 protein is isolated.

Suitably the GRAF1 protein is purified.

Suitably the GRAF1 protein is recombinant.

Suitably the GRAF1 protein is in vitro.

Molecular Structure of GRAF proteins

GRAF1 has four predicted domains and the function of each of these domains was dissected. Two of these domains (BAR and PH domains) were shown to act together to precisely stabilise the high curvature of the tubular endocytic membranes which GRAF lines. The other two domains (GAP and SH3 domains) were shown to mediate interactions with other proteins. Several biochemical and biophysical techniques were used to identify novel proteins which also act in this endocytic pathway. The major interacting partner of the GRAF1 SH3 domain was shown to be Dynamin, which was also shown to be necessary for GRAF1-dependent endocytosis. Interestingly, the interacting partners of GRAF1 fell into three groups based on what was known about their functions: proteins implicated in endocytosis, proteins implicated in the regulation of small G-proteins (master regulators of cell morphology), and proteins implicated in focal adhesion disassembly. Focal adhesions are regions of the cell where it sticks tightly to its surroundings. At these sites the extracellular matrix, that surrounds cells in tissues, is connected to Actin filaments that form the cell cytoskeleton and determine cellular morphology. These focal adhesions represent an important interaction of the cell with its environment and rely on the ligation of transmembrane integrins. These sites are constantly remodelled and, when the cell migrates, these tight connections must be disassembled at the rear of the cell, and new adhesion points must form at its front. In addition to adhesion site turnover and important changes in the cytoskeleton, a migrating cell must deliver membranes to its front to allow this side of the cell to elongate in the direction of migration. Since the studies summarised above implicated GRAF1 in all of the basic processes necessary for cell migration, it was hypothesised that this might place GRAF1 in an ideal position to coordinate cell morphology and migratory events. The level of GRAF1 in cells was shown to correlate with types of cell morphologies. It was then shown that GRAF1-dependent endocytosis occurs preferentially from adhesion sites. This provided the first evidence in non-neuronal cells for specific plasma membrane regions from which endocytosis preferentially occurs. By specifically depleting levels of GRAF1 it was shown that this protein is necessary for adhesion sites to be disassembled and indeed, in the absence of GRAF1 (either at the level of the gene or the protein), cells were incapable of migration. A model for GRAF1-dependent adhesion site disassembly has been produced from the synthesis of these results. An evolutionary study of GRAF1-like proteins in many species was also performed, and accompanying studies on the close relatives of GRAF1 in mammals were carried out. Results strongly suggested that such proteins may function in similar ways to GRAF1. These studies have revealed novel, extensive and dynamic cellular anatomy responsible for the coupling of endocytosis and cell migration. The importance of membrane trafficking, adhesion site disassembly and cytoskeletal changes in cell migration have been hotly debated for decades. These studies have shown that the three processes are intricately linked and interdependent, and provide insight into why interdisciplinary approaches are required and how they might be approached for future studies of cell migration. They also show how a single protein is necessary to couple these events. An important outcome of this research is to suggest how changes in membrane trafficking pathways can result in the diseases associated with aberrant amounts of GRAF1 and similar proteins. These studies provided a range of novel tools that can be used for the further study of the GRAF1-dependent endocytic pathway and its role in disease. These new tools include a further marker of the endocytic membranes of the pathway (Rab8), methods to preserve the fragile membranes of this pathway, methods that can be used to specifically inhibit the pathway, and a small molecule that can be used to stimulate the pathway. The latter could potentially be used as a non-toxic therapeutic agent in the treatment of human leukaemias and inhibitors of this pathway would also be predicted to inhibit cancer cell invasion and metastasis as well as provide the basis for novel and cleaner immunosuppressive therapies.

### Agent

The candidate modulators according to the present invention may comprise any suitable entity which might be capable of affecting GRAF protein activity. For example, the candidate modulators may be chemical or biochemical entities or agents. As used herein, the term “agent” may be a single entity or it may be a combination of entities. Suitably, the agent modulates the activity of GRAF.

The agent may be an antagonist or an agonist of GRAF. Suitably, the agent is an inhibitor of GRAF, such as an inhibitor of GRAF GAP activity.

The agent may be an organic compound or other chemical. The agent may be a compound, which is obtainable from or produced by any suitable source, whether natural or artificial. The agent may be an amino acid molecule, a polypeptide, or a chemical derivative thereof, or a combination thereof. The agent may even be a polynucleotide molecule—which may be a sense or an anti-sense molecule. The agent may even be an antibody.

The agent may be designed or obtained from a library of compounds, which may comprise peptides, as well as other compounds, such as small organic molecules.

By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetic, a derivatised agent, a peptide cleaved from a whole protein, or a peptide synthesised synthetically (such as, by way of example, either using a peptide synthesiser or by recombinant techniques or combinations thereof, a recombinant agent, an antibody, a natural or a non-natural agent, a fusion protein or equivalent thereof and mutants, derivatives or combinations thereof).

Typically, the agent will be an organic compound. Typically, the organic compounds will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the agent comprises at least one cyclic group. The cyclic group may be a polycyclic group, such as a non-fused polycyclic group. For some applications, the agent comprises at least the one of said cyclic groups linked to another hydrocarbyl group.

The agent may contain halo groups, for example, fluoro, chloro, bromo or iodo groups.

The agent may contain one or more of alkyl, alkoxy, alkenyl, alkylene and alkenylene groups—which may be unbranched- or branched-chain.

The agent may be in the form of a pharmaceutically acceptable salt—such as an acid addition salt or a base salt—or a solvate thereof, including a hydrate thereof. For a review on suitable salts see Berge et al, (1977) J. Pharm. Sci. 66, 1-19.

The agent of the present invention may be capable of displaying other therapeutic properties.

The agent may be used in combination with one or more other pharmaceutically active agents.

If combinations of active agents are administered, then they may be administered simultaneously, separately or sequentially.

Agents may exist as stereoisomers and/or geometric isomers—e.g. the agents may possess one or more asymmetric and/or geometric centres and so may exist in two or more stereoisomeric and/or geometric forms. The present invention contemplates the use of all the individual stereoisomers and geometric isomers of those agents, and mixtures thereof.

Agents may be administered in the form of a pharmaceutically acceptable salt.

Pharmaceutically-acceptable salts are well known to those skilled in the art, and for example include those mentioned by Berge et al, (1977) J. Pharm. Sci., 66, 1-19. Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

When one or more acidic moieties are present, suitable pharmaceutically acceptable base addition salts can be formed from bases which form non-toxic salts and include the aluminium, calcium, lithium, magnesium, potassium, sodium, zinc, and pharmaceutically-active amines such as diethanolamine, salts.

A pharmaceutically acceptable salt of an agent may be readily prepared by mixing together solutions of the agent and the desired acid or base, as appropriate. The salt may precipitate from solution and be collected by filtration or may be recovered by evaporation of the solvent.

The agent may exist in polymorphic form.

The agent may contain one or more asymmetric carbon atoms and therefore exists in two or more stereoisomeric forms. Where an agent contains an alkenyl or alkenylene group, cis (E) and trans (Z) isomerism may also occur. The present invention includes the individual stereoisomers of the agent and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of the agent may also be prepared from a corresponding optically pure intermediate or by resolution, such as by H.P.L.C. of, the corresponding racemate using a suitable chiral support or by fractional crystallisation of the diastereoisomeric salts formed by reaction of the corresponding racemate with a suitable optically active acid or base, as appropriate.

The agent may also include all suitable isotopic variations of the agent or a pharmaceutically acceptable salt thereof. An isotopic variation of an agent or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Inflated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent and pharmaceutically acceptable salts thereof of this invention can generally be prepared by conventional procedures using appropriate isotopic variations of suitable reagents.

It will be appreciated by those skilled in the art that the agent may be derived from a prodrug. Examples of prodrugs include entities that have certain protected group(s) and which may not possess pharmacological activity as such, but may, in certain instances, be administered (such as orally or parenterally) and thereafter metabolised in the body to form the agent which is pharmacologically active.

It will be further appreciated that certain moieties known as “pro-moieties”, for example as described in “Design of Prodrugs” by H. Bundgaard, Elsevier, 1985 (the disclosure of which is hereby incorporated by reference), may be placed on appropriate functionalities of the agents. Such prodrugs are also included within the scope of the invention.

### Assaying GRAF Activity

Suitably GRAF activity is assayed by determining the GAP activity of the GRAF of interest. In other words, GRAF activity is suitably assayed by determining the ability of GRAF protein to activate GTPase(s).

GRAF has activity on RhoA and Cdc42 so can be assayed against either. RhoA and Cdc42 may be used separately or simultaneously in assays of the invention, suitably separately.

Thus the invention also relates to diverse small molecule screening for inhibitors of the GAP activity of GRAF1 .

GAP assays are routinely used and may be conveniently developed for HTS. Fluorescent readouts may be used as are known in the art (e.g. Reza Ahmadian et al. see Eberth et al. Biol Chem November 2005). Such assays use tamraGTP which senses conformational changes in small G-proteins induced upon nucleotide hydrolysis.

### Tamra-GTP Hydrolysis Assays:

Tamra-GTP hydrolysis assays may be performed using 0.2 uM tamra-GTP (Eberth et al., Biol. Chem., 2005), 0.2 uM purified RhoA and/or Cdc42, 1 uM of the purified stimulant GAP protein (i.e. GRAF protein of the invention).

The assays may be conducted with or without purified protein additive proteins or small molecules depending, upon the treatment/study being undertaken. Each hydrolysis reaction is measured using a stopped-flow spectrophotometer at 25 degrees Celsius in a buffer comprising 30 mM Tris/HCl (pH 7.5), 10 mM KH2PO4/K2HPO4 (pH 7.4), 10 mM MgCl2, and 3 mM DTT. Tamra-GTP is excited at 546 nm, with emission being recorded at 570 nm using a cut-off filter.

Verification of small molecules as inhibitors of GRAF protein GAP activity may be performed in cells using the ability of GRAF protein overexpression to induce morphological change as a readout.

### Optional Assay Conditions

Cofactors or other relevant materials may be supplemented into the assays of the invention. For example, membranes or membrane components may be included if desired.

GRAF protein may be supplied in a purified form.

GRAF protein may be supplied in a tubular form.

GRAF protein may be complexed with dynamin and/or GIT1 and/or FAK and/or PAK2.

GRAF protein may be complexed with dynamin and/or GIT1 and/or PAK2 and/or synaptojanin and/or caskin1

Suitably the assays of the invention are in vitro assays.

Suitably the GTPase substrate for assay of GAP activity is RhoA or Cdc42. Suitably the GTPase substrate is RhoA, which has the benefit of being a major target of GRAF1 in vivo.

### Optional Assay Steps

Downstream validation is advantageously conducted on candidate modulators or other agents identified according to the methods of the invention. Such validation may take the form of further test(s) to determine the effect (if any) of the modulators or agents on other aspects of GRAF function as discussed below.

In one embodiment the method further comprises the step of carrying out an endocytic assay. This has the advantage of verifying putative inhibitors in vivo.

In one embodiment the method further comprises the step of carrying out an adhesion assay. This has the advantage of verifying putative inhibitors in vivo.

In one embodiment the method further comprises the step of carrying out a selectivity assay. Such assays may be easily assembled by the skilled operator. For example, it may be desired to test via ELISA the effects on global small G-protein balance induced upon treatment with the putative inhibitor.

HeLa cells are particularly useful in endocytic/adhesion models.

Supplementary tests may also be carried out in whole animal models for example by looking at the ability to fight infection or to inhibit tumour progression.

In vivo models on molecules identified and verified in vitro are available. For example, there are many inducible and spontaneous murine cancer models, as well as murine immune effector cell activity assays.

In more detail, assays may take one of the following examples of suitable formats:

In one embodiment the method further comprises the step of assaying for modulators of FAK activity in vitro. Specific examples of such a step may comprise:
Using in vitro phosphorylation assays with FAK substrates
Assay with radioactive phosphate
Verified (in terms of specificity) by testing effects on other tyrosine kinases
Verified in cells

In one embodiment the method further comprises the step of assaying for modulators of GRAF RhoGAP activity in vitro. Specific examples of such a step may comprise:
Using in vitro assays to quantify effects on the ability of GRAF proteins to stimulate hydrolysis of GTP by Rho family small G-proteins; fluorescence-based (tamra-GTP or equivalent).
Verified (in terms of specificity) by testing effects on other GAP domain containing proteins and their abilities to stimulate small G-protein GTP hydrolysis.
Verified in cells.

In one embodiment the method further comprises the step of assaying for modulators of GRAF-FAK interaction in vitro. Specific examples of such a step may comprise:
Using in vitro protein-protein interaction assays
Verified (in terms of specificity) using other SH3 domains as pulldown baits
Verified in cells

In one embodiment the method further comprises the step of assaying for changes in GRAF distribution in cells (e.g. by examination, e.g. in vivo). Specific examples of such a step may comprise: microscopy-based examination.

In one embodiment the method further comprises the step of assaying for specific modulators of endocytic routes in vivo. Specific examples of such a step may comprise: microscopy-based examination with specific markers of clathrin-dependent, caveolar, GRAF-mediated, and clathrin-, caveolae- and GRAF-independent endocytosis.

### Endocytic Assay:

Ligands may be used in a cell biological screen for small molecular inhibitors of GRAF-dependent endocytic pathways, for example:

a. Transferrin (clathrin-mediated endocytosis)
b. Epidermal growth factor (clathrin-mediated and clathrin-independent endocytosis)
c. Dextran (predominantly clathrin-independent endocytosis)
d. SV40 virions (clathrin-independent endocytosis)
e. Cholera Toxin B subunit (clathrin-mediated and clathrin-independent endocytosis
f. Shiga Toxin B subunit (clathrin-mediated and clathrin-independent endocytosis). Small molecules that affect uptake or trafficking of b/c/d/e/f but leave (a) intact are identified (or verified) as putative inhibitors of GRAF-mediated endocytosis/trafficking.

In one embodiment a negative control is used as a further reference sample. In this embodiment suitably Arf6 is used.

Adhesion assays may also be used to further validate target(s) identified by the methods of the invention.

### Adhesion Assay:

Cells are dissociated from substrate in EDTA-based buffer in the presence of small molecule (present throughout from this point), and replated onto 96 well plates for 5/15/30/60/180/720 minutes before washing and addition of a vital (coloured) dye. Cells stuck down are then lysed and the number of living, adherent cells is indirectly measured spectrophotometrically. This may be normalised to total protein concentration in each well after lysis.

### Manufacture of GRAF Protein

GRAF protein may be manufactured by any suitable technique known to those skilled in the art. GRAF protein may be purified or isolated from natural or recombinant sources. GRAF protein may be made synthetically by in vitro translation or by chemical synthetic means. Any of the numerous commercially available protein synthesis services may be used to make GRAF protein for use in the invention.

### Further Applications

In another aspect, the invention relates to a method for identifying an agent that modulates cell migration and/or focal adhesion disassembly comprising the steps of:

(a) contacting GRAF1 with an agent; and
(b) determining if said agent modulates GRAF-1 dependent endocytosis,
wherein modulation of GRAF-1 dependent endocytosis by said agent is indicative that said agent modulates cell migration and/or focal adhesion disassembly.

GRAF-1 dependent endocytosis may be monitored using any suitable technique known to the skilled worker, such as using either DiI or dextran, before staining with GRAF1. GRAF-1 dependent endocytosis may be monitored using immunofluorescence and/or a quantitative fluorometric assay.

The invention also relates to methods for modulating cell migration in vivo or in vitro comprising the use of GRAF1, such as the use of GRAF1 for modulating cell migration in vivo or in vitro.

The invention also discloses for the first time the scientific rationale for GRAF1 being a tumour suppressor protein (in terms of reduced uptake of adhesion receptors, increased adhesion to tumour cell niche etc.)

The invention also related to use of Rho kinase inhibitor (or any other activators of the pathway) as another novel therapeutic rationale.

In another aspect, the invention relates to a method for, (or use of GRAF protein for), localising a protein to a plasma membrane comprising PtdIns(4,5)P2 in vivo or in vitro comprising the use of GRAF1. Suitably the PtdIns(4,5)P2 may be present as a liposome, a tubule, a focal adhesion membrane, or at the leading edge of migratory cells.

GRAF1 may also be used for regulating the formation of tubular structures in vivo or in vitro.

### INDUSTRIAL APPLICATION

Clearly the invention finds application in treating or preventing disease comprising modulating the activity and/or expression of GRAF1 in a subject, and/or the use of GRAF-1 in the manufacture of a medicament for the treatment of disease, as well as in study and screening applications connected to same. Relevant diseases include mental retardation and cancer, particularly solid cancers such as tumours.

The methods of the invention may be applied in, screening for modulators of GRAF1 with therapeutic potential, such as high throughput screening. Especially suitable are methods involving the GAP activity of GRAF protein such as GRAF1, i.e. the ability of GRAF protein to activate GTPases.

In addition, the inactive mutant of GRAF1 disclosed herein (R412D), ablates the GAP domain activity, and finds utility as a control in the methods of the invention.

It is disclosed that GRAF1 regulates turnover of focal adhesions. GRAF therefore has a dual role in haematological malignancies (where is a tumour suppressor) and solid cell malignancies (where mutants can cause cellular invasion/metastases). Inhibitors of GRAF1 have application in treating invasive cancers.

Furthermore, inhibitors of GRAF find application as immunosupressants (by blocking bacterial toxin and viral entry endocytic pathways).

The invention may be applied to numerous medical indications, including for the treatment of invasive solid organ malignancies, and for specific immunosuppression.

Modulators of, or screening methods based upon, GRAM and OPHN have industrial application in disease for the reasons given above. Furthermore, GRAF2 and GRAF3 are considered to be oncogenic, and it is thus desirable to target them for different diseases depending on tissue/cell type expression/distribution which can be advantageously determined by the operator.

### Membrane Biology

There are several families of proteins known to be capable of deforming flat membranes and stabilising highly curved membranes. These are therefore potential candidates for roles in producing the plasma membrane invaginations required for endocytosis. Members of one such family of proteins are predicted to include a region known as a BAR domain, which dimerises to form a banana-shaped module that can sense and generate highly curved membranes. Many BAR domain-containing proteins are multidomain in nature. One subfamily of BAR domain-containing proteins includes GRAF1 (for GTPase Regulator Associated with Focal adhesion kinase) and Oligophrenin 1. GRAF1 appears to be an important protein in white blood cells, since mutation of the gene encoding GRAF1, or a reduced level of GRAM, has previously been associated with malignant leukaemias in human patients. Furthermore, GRAM levels are significantly increased in malignant lung cancers. The gene encoding a close relative of GRAF1, Oligophrenin1, has frequently been found to be mutated in patients with mental retardation and levels of the protein encoded by this gene are increased in gastrointestinal malignancies. Little is known about this protein family despite the obvious importance of understanding the normal function of these proteins: understanding these normal functions will allow the development of a platform from which the important diseases associated with their malfunction could be understood. GRAF1 is known to interact with a protein (Focal Adhesion Kinase) that appears to be necessary for at least one Clathrin-independent endocytic pathway. The main hypotheses of this dissertation were that GRAF1 might act to produce membrane deformation within one or more of these endocytic pathways, and that study of its normal function would contribute to the understanding of disease processes involving dysregulation of GRAF1 and its close protein relatives. In this dissertation a wide variety of biochemical, biophysical and cell biological techniques were employed to analyse the function of GRAF1 in mammalian cells. To determine the localisation of GRAM, antibodies that specifically recognise this protein were produced and used to detect endogenous GRAF1 in human cells. This approach, coupled with analytical cell biology, revealed an extensive, and novel, system of tubular membranes lined by GRAF1. These appeared to communicate directly with both the cell periphery and distant intracellular membrane-bound compartments. These membranes were shown to be endocytic in nature, and capable of internalising extracellular fluid and GPI-linked proteins, as well as the bacterial toxins responsible for the important human diseases cholera and dysentery. These endocytic tubules did not use Clathrin, or other proteins (Caveolin1 and Flotillin1) that have been implicated in Clathrin-independent endocytic events. Using GRAF1 tagged with a fluorescent marker, the distribution of this protein was examined in living cells in real time. Surprisingly, these tubules were shown to be highly dynamic, undergoing turnover on a much faster time-scale than has been observed for other endocytic events. To determine if GRAF1 was necessary for the formation and function of the endocytic tubules that it lines, cells were then depleted of GRAF1. In the absence of GRAF1, the amount of endocytosis into tubular membranes was profoundly reduced, yet this treatment did not disrupt endocytosis via Clathrin mediated mechanisms. Further analysis revealed that GRAF1-dependent endocytosis and Clathrin-mediated endocytosis each accounted for around half of plasma membrane turnover in these cells, and that cells depleted of both types of endocytosis did not survive. These discoveries allowed, for the first time, an extensive biochemical and biophysical interrogation of the prevalent endocytic pathway that is dependent on GRAF1.

### GRAF Biology

GRAF1 is a BAR, PH, GAP, and SH3 domain-containing protein which we show interacts with proteins involved in the disassembly of focal complexes and adhesions via its SH3 domain. We have shown that it regulates a major clathrin-independent endocytic pathway which is responsible for the disassembly of these adhesions and thereby coordinates cellular migratory events. The GAP domain of GRAF1 has GTPase activating activity for RhoA and Cdc42 and we believe that RhoA is its major target in vivo. The GAP activity of GRAF1 is necessary for the function of the protein, and the local changes in small G-protein balance are thus necessary for the endocytic disassembly of adhesive contacts.

Functional GRAF1 expression is reduced or lost in the bone marrow of acute myeloid leukaemia/myelodysplastic syndrome patients either through translocation, mutation, deletion, or through promoter methylation. FAK, which is involved in the regulation of GRAF1-dependent processes, is upregulated in solid organ malignancies, including breast cancer.

Known approaches to inhibiting cellular migration and cancer cell invasion are rather unspecific and interfere with a large number of pathways. We show how a single protein is necessary for this cellular migration and thus focussing on this protein as a target provides much greater specificity than traditional approaches. The activity of the GAP domain of GRAF1 is key to its biological function and we have found that a single amino acid change in the GAP domain of this protein blocks the pathway. A cell-permeable inhibitor of this GAP activity would therefore have similar effects—thus suitably candidate modulators of the invention are cell permeable. Of course, side effects might be expected on migrating cell type(s) in adult tissue. However, this mechanism of cellular migration is, we believe, limited to mature effector immune cells, cells in healing wounds, and cancer cells. Interfering with these processes allows the drug (candidate modulator) to act as an immunosuppressive, anticancer agent.

We disclose evidence from a knockout cell line that these cells do not migrate in culture. In terms of disease association, the evidence is very strong that increased activity of the pathway promotes cellular migration/cancer cell invasion.

Activation of the target process by application of a Rho kinase inhibitor gives the predicted phenotype, further supporting our approach.

Since endocytic events require membrane sculpting molecules, and since BAR domain-containing proteins are capable of generating and stabilising membrane curvature in distinct locations in the cell, such a protein may be required to fill such a role in Clathrin-independent endocytic events, and identification and analysis of such a protein should provide insight into the cell biological role of the endocytic event that it regulates. Further, since FAK was shown to be shown in a kinome screen to be necessary for a portion of Clathrin-independent endocytic events, GRAF1 (which interacts with this kinase) presented the most likely candidate from the BAR domain-containing protein family to be involved in Clathrin-independent endocytosis. The results presented herein describe GRAF1 as the first clearly-defined, non-cargo marker of the CLIC/GEEC endocytic pathway, and demonstrate that GRAF1 is necessary for this process to proceed. Through a domain-by-domain dissection of GRAF1, the first mechanistic insights into this prevalent endocytic pathway have been revealed. GRAF1 localises to PtdIns(4,5)P2-enriched tubular and punctate membranes in vivo via its N-terminal BAR and PH domains, and its SH3 domain directly binds the membrane scission protein Dynamin which is required for the CLIC/GEEC pathway of endocytosis. GRAF1 also binds proteins implicated in the disassembly of adhesion sites. Using this knowledge it was shown that the CLIC/GEEC endocytic pathway emanates preferentially from adhesion sites, and that GRAF1 allows these to be disassembled in order for cellular migration to proceed. These studies have thereby revealed novel and dynamic cellular anatomy responsible for the coupling of endocytosis and cell migration.

### GRAF1 and Endocytic Membranes

The study of Clathrin-independent mechanisms of endocytosis has been severely restricted by a frustrating lack of clarity as to how such processes can proceed at the molecular level. Despite these limitations, many cargoes have been shown to be internalised by these mechanisms, and much information regarding the upstream lipid compositions that are permissive for these pathways to proceed, as well the sensitivity of these pathways to drugs, has been elucidated4. Further, the discovery that Flotillin1 and Caveolin1 are involved in the endocytosis of cargoes via Clathrin independent mechanisms has contributed significantly to the field. It is clear that Clathrin-independent endocytic mechanisms are difficult to study and, since they are linked to the physiology of membrane microdomains, such difficulty may stem from our incomplete understanding of cellular lipid homeostasis. It may also stem from a lack of hub proteins analogous to the CME regulator AP2, which has allowed the identification of myriad proteins involved in Clathrin-mediated endocytosis, as well as helping to determine how this process is coordinated in time and space at the molecular level36. Despite the prevalence of CLIC/GEEC endocytosis, previous studies interrogating this pathway have been limited to the elucidation of the cargoes that it is capable of internalising, the morphologies of its early carriers, and its dependence on cellular Actin and small G-proteins. Such research has presumably been severely hampered by the stringent (non-standard) fixation procedures that are shown here to be required for the effective preservation of the tubular membranes of this pathway. Here it was also shown that GRAF1 is necessary for endocytosis through the CLIC/GEEC endocytic pathway. In the absence of GRAF1, not only can internalisation through this pathway not proceed, but the tubular membranes of this pathway are not present. The BAR and PH domains of GRAF1 comprise a lipid binding module that acts as a ‘coincidence detector’, binding preferentially to highly-curved membranes that contain the plasma membrane-enriched phosphoinositide PtdIns(4,5)P2. In addition to membrane curvature sensing, this module can generate membrane curvature in vitro. A spectrum exists between the membrane curvature sensing and generating capabilities of BAR and N-BAR domains. The GRAF1 BAR domain does not include putative amphipathic helices (as are found in N-BAR domains) and is a relatively weak membrane tubulator in vitro. The overexpression of GRAF1 BAR+PH in vivo does not produce as extensive a membrane tubulation phenotype as that observed with N-BAR overexpression. Instead, this protein binds to and specifically stabilises the early endocytic carriers of the CLIC/GEEC pathway through reversible binding. It is therefore likely that the role of the BAR and PH domains of GRAF1 is to specifically stabilise (rather than generate de novo) the high curvature of the tubular membranes of this pathway that has been first produced by upstream processes. The nature of these upstream processes is unknown but may include the generation of spontaneous membrane curvature through the accumulation of specific lipids with appropriate stereometries. They might also be dependent on small G proteins or other proteins capable of generating membrane curvature such as N-BAR or F-BAR domain-containing proteins. A question remains as to how GRAF1 might be removed from CLIC/GEEC endocytic membranes once its function has been performed. The small G-protein Arf6 is also implicated in endocytosis through the CLIC/GEEC pathway. It is known that hydrolysis of GTP is required for Arf6's role in endocytic cycling. GRAF1 is found in a complex with the Arf6 GAP GIT1, which would favour GIP hydrolysis by Arf6. Since active (GTP-bound) Arf6 produces a positive-feedback cycle resulting in rises in PtdIns(4,5)P2 levels at the plasma membrane, activation of Arf6 GTP hydrolysis by GIT1 would reduce PtdIns(4,5)P2 levels in endocytosing membranes. GRAF1 also binds the lipid phosphatase Synaptojanin1, which also catalyses dephosphorylation of PtdIns(4,5)P2. GRAF1 binds PtdIns(4,5)P2-containing membranes. In order for GRAF1 to come off (and not rebind to) membranes of the CLIC/GEEC endocytic pathway, it is likely that PtdIns(4,5)P2 levels need to be reduced. Once sufficient GRAF1 has bound to CLIC/GEEC membranes via its N-terminus, and endocytosis has occurred, the recruitment of Synaptojanin1 and GIT1 by other domains may allow the kinetics of its association and dissociation to change in a regulated manner, such that GRAF1 is removed from the membrane.

### GRAF 1, Small G-Proteins and Membrane Trafficking

The links between Rho family small G-proteins and endocytic regulation have been reviewed. The studies presented here may provide some clarity as to why overexpressed mutants of this family have endocytic phenotypes. It possible that these proteins have no direct role in the membrane deformation events that characterise endocytosis per se, but given their critical role in adhesion site and cytoskeletal regulation, these may be permissive for endocytic events that preferentially occur from specific regions of the plasma membrane. While the CLIC/GEEC pathway is Cdc42-dependent, and acute inhibition of Rho Kinase increases endocytosis through this route; without RhoA activity in the longer term adhesion sites would not be capable of maturing and therefore the large clusters of plasma membrane lipids at adhesion sites that are permissive for the CLIC/GEEC pathway would not exist. Care should therefore be taken when using the overexpression of dominant-negative Rho family mutants to determine the small G-protein dependence of particular endocytic pathways since data derived from these methods may be uninterpretable as Rho family members may be distantly involved in producing the phenotypes in question. The small G-protein Arf6 is capable of binding to lipid membranes and induces deformation of these. This is dependent on the function of an N-terminal amphipathic helix which likely inserts into the membrane in a manner analogous to a similar helix in N-BAR domains. Arf6 T27N overexpression appears to inhibit the CLIC/GEEC pathway, blocking Cholera Toxin traffic through this route to the Golgi apparatus, although the toxin is still found in Arf6 T27N-positive endocytic carriers of the CLIC/GEEC pathway9. This suggests that an early step in the CLIC/GEEC endocytic pathway is Arf6-dependent and it is possible that this protein induces the initial curvature of the endocytic tubules that require stabilisation by GRAF1. Interestingly, the Arf6 GAP protein GIT1 has been implicated in trafficking between the plasma membrane and endosomes175. GRAF1 in found in a complex with GIT1, which may provide a biochemical link to Arf6. The early endocytic membranes of the CLIC/GEEC pathway were shown to be Rab8-positive, consistent with previous data supporting a role for Rab8 in a similar membrane trafficking pathway. Each member of the Rab family of small G-proteins appears to regulate a distinct membrane trafficking step although the precise mechanisms by which they perform this are unknown. Rab8 has previously been shown to be associated with ‘macropinocytic’ structures and Arf6-associated tubular membranes that emerge from these. Furthermore, 61 integrins have been found in Rab8-positive tubules and trafficking of 61 integrins is also Arf6-dependent184. Rab8 has also been shown to be necessary for Cholera Toxin delivery to the Golgi. Taken together these observations suggest that GRAF1, Rab8 and Arf6 work together in the regulation of the CLIC/GEEC pathway. Further studies will directly address the connections between these proteins.

### GRAF1 and Dynamin

GRAF1 binds directly to Dynamin and is found with Dynamin at the cell surface (but not on CLIC/GEEC endosomal membranes), suggesting a role for this protein in the scission of CLIC/GEEC pathway endosomes from the plasma membrane. It has previously been suggested that the CLIC/GEEC pathway is Dynamin-independent since overexpression of Dynamin K44A (which is deficient in nucleotide hydrolysis) allows apparent CTxB internalisation. However, this ‘internalised’ CTxB colocalises with Dynamin K44A in tubular compartments. At least a subset of Dynamin K44A-positive tubules are known to be connected to the plasma membrane and any CTxB trapped in these tubules may be partially inaccessible to even stringent washing conditions. It is also unclear whether Dynamin K44A-positive tubules are actually ‘trapped’ compartments incapable of progression, or induced structures. Such overexpression experiments necessarily require a rather long period before studies can be performed and, in Dynamin K44A-overexpressing cells, apparent Dynamin-independent endocytic pathways may be functionally-upregulated. To clarify this issue of Dynamin-dependence, cells were treated acutely with dynasore, a cell-permeable inhibitor of Dynamin function. In addition to a loss of tubular endocytosis through the CLIC/GEEC pathway, GRAF1 was found to redistribute from tubular endocytic membranes to basal complexes. Consistent with this, in Dynamin K44A-expressing cells, CTxB delivery to the Golgi—the likely destination of CLIC/GEEC endocytic membranes—was profoundly inhibited9. Taken together, these data strongly suggest that the CLIC/GEEC endocytic pathway is indeed Dynamin dependent, although it may have a complex role in this process. It may be addressed if a specific splice variant is responsible, and immuno-electron microscopy will be used to discover its spatiotemporal distribution in the CLIC/GEEC endocytic pathway.

### GRAF1, Caveolae and Caveolae-Like Structures

The lack of specific markers has produced great difficulty in completely distinguishing between Clathrin-independent endocytic pathways by electron microscopy. The CLIC/GEEC endocytic pathway was previously been shown to include both Caveolin1-positive and -negative structures. The results herein show that GRAF1-dependent endocytosis is Caveolin1-independent suggesting refinement of the definition of the CLIC/GEEC pathway to include only Caveolin1-negative membranes. The author suggests that this route of endocytosis is better defined as GRAF1-dependent endocytosis. This pathway is not marked by Flotillin1, which has also been implicated in endocytosis from caveolae-like structures. It is yet to be determined if tubular pathways that are Clathrin-, Caveolin1- and GRAF1-independent exist, although at least in HeLa cells very few or no tubular endocytic membranes can be discerned upon GRAF1 depletion. Further, CLIC/GEEC-like pathways might also be observed in cell types ordinarily deficient in GRAF1 and may be lined by other BAR domain-containing proteins of the GRAF family such as GRAF2 or OPHN1.

### GRAF1 and the Cytoskeleton

The CLIC/GEEC endocytic pathway is dependent on both, the integrity of both F-Actin and microtubule cytoskeletal networks. GRAF1 co-immunoprecipitates with á-Actinin (a protein that cross-links F-Actin filaments), and may therefore provide a direct link between CLIC/GEEC membranes and the Actin cytoskeleton. The Rho family of small G-proteins is extensively implicated in regulation of both Actin and microtubule cytoskeletons and GRAF1 has an active RhoGAP domain. Inhibition of the major RhoA effector, Rho Kinase, acutely increases endocytosis through the CLIC/GEEC pathway, suggesting that activity of this kinase ordinarily inhibits this pathway. The GRAF1 interactome includes several other proteins that are capable of small G-protein regulation, including PAK2 and PIX which favour Cdc42 and Rac1 over RhoA activity. While RhoA is essential for the assembly and maintenance of mature focal adhesions, Cdc42 activity is necessary for the CLIC/GEEC endocytic pathway and active Cdc42 colocalises with GRAF1. Hence, a change in active small G-protein balance from RhoA to Cdc42 may occur via the action of GRAF1 and its interacting proteins to allow the loss of focal adhesion assembly and maintenance signals (through RhoA inactivation) concomitantly with permissive signals through Cdc42 to allow CLIC/GEEC pathway progression. The GRAF1 interacting protein GIT1 negatively regulates Arf6. In addition to a role in membrane trafficking, Arf6 is also capable of regulation of the actin cytoskeleton, and this is likely via its ability to produce large rises in PtdIns(4,5)P2 levels at the plasma membrane which regulates WASP and Profilin activity. Arf6 activity is sufficient to induce membrane ruffle formation and may recruit Rac1 to these sites to further induce changes in the local cytoskeleton. Other proteins are implicated in directly linking membranes to cytoskeletal elements and may play roles early in the CLIC/GEEC endocytic pathway. For example, the F-BAR domain-containing proteins Toca1, Cip4 and FBP17 can interact directly with both membranes and the actin cytoskeleton. The BAR domain-containing protein SNX9 is necessary for Clathrin-mediated endocytic events, and has recently been shown to be necessary for about 60% of fluid phase uptake in mouse kidney epithelial (BSC1) cells. It appears that this protein is associated not only with Clathrin-coated pits, but also with circular dorsal ruffles308 which are known to participate in macropinocytic, Clathrin independent endocytosis from the dorsal surface of the plasma membrane. SNX9 associates with N-WASP, stimulating N-WASP/Arp2/3-mediated Actin polymerisation. The PX domain of SNX9 binds to PtdIns(4,5)P2 and, together with the BAR domain, may play a role in linking Actin assembly to membrane deformation at the plasma membrane with little discrimination between endocytic routes. The early carriers of the CLIC/GEEC endocytic pathway appear to be Actin-dependent88, and a functional homologue of SNX9 (or even GRAF1 itself), may similarly link Actin assembly to the initial membrane deformation in this pathway. There are no biochemical links known between GRAF1 and SNX9. Interestingly however, SNX9 colocalises with GFP-GPI puncta on the basal surface of the cell (although it has not been shown if this is linked to GFP-GPI endocytosis), suggesting that this protein may play a role upstream of GRAF1 in the CLIC/GEEC pathway. Perhaps this protein is involved in non-specific (but direct) linkage of membrane microdomains and their associated proteins that are permissive for endocytosis to F-Actin.

### GRAF1 and Adhesion Site Disassembly

In addition to a lack of mechanistic insight into Clathrin-independent endocytic pathways, it is clear that we also do not understand why such pathways are required in vivo and therefore what makes them functionally distinct from Clathrin-mediated mechanisms. The results herein indicate the first cell physiological function for a Clathrin-independent endocytic pathway. In the absence of GRAF1, and therefore endocytosis through the CLIC/GEEC pathway, focal adhesion disassembly cannot occur. Furthermore, CLIC/GEEC endocytic tubules arise from sites of adhesion, and GRAF1 is found at disassembling focal complexes with markers of focal adhesion disassembly. When focal adhesion disassembly is acutely stimulated, the amount of endocytosis through the CLIC/GEEC pathway increases. Furthermore, GRAF1 can be found to colocalise with â1-integrin in puncta and tubules. â1-integrin is known to be trafficked in a Rab8-positive tubules and is Arf6-dependent, and these proteins respectively mark and are necessary for the CLIC/GEEC endocytic pathway. In the absence of GRAF1 cells have more focal adhesions than normal, and therefore more integrins on their surfaces. Taken together, these results strongly suggest that the CLIC/GEEC endocytic pathway is necessary for adhesion site disassembly and that this may occur through endocytosis of adhesion proteins, including integrins and GPI-linked proteins, which enter via this pathway. Focal adhesion disassembly has previously been shown to be dependent on microtubules and Dynamin. Consistent with these findings, the early CLIC/GEEC endocytic carriers are microtubule-dependent, and endocytosis through this pathway is dependent on Dynamin. It has previously been suggested that focal adhesion disassembly occurs after the growing end of microtubules deliver some ‘releasing factor’ to adhesion sites and that Dynamin plays an atypical role at these locations. While these suggestions were consistent with published findings, a new model must now be proposed as to how adhesion site disassembly occurs. For simplicity, the model presented below ignores several biochemical events known to be necessary for focal adhesion disassembly, but to the best of the author's knowledge is entirely consistent with all published data in this field. It is first necessary to re-examine focal adhesions and their formation. This has previously been considered a simple signalling process from ligated integrins to the interior of the cell to build up a protein complex that links the integrins to the Actin cytoskeleton. This is almost certainly a gross underestimation of the complexity of the process. It is known that at least several of the glycolipid receptors for bacterial exotoxins are enriched in adhesion sites. Furthermore, it has been shown that a large proportion of plasma membrane microdomains are regulated by adhesion to surrounding matrix components. This is likely due to downstream effects from the clustering of integrins by extracellular matrix components. The transmembrane domains of integrins themselves may bind specific microdomain-associated lipids preferentially, and thus clustering of these proteins by matrix binding may allow the initial formation of a membrane microdomain. Alternatively, since other microdomain-associated proteins might also be clustered by matrix, (for example GPI-linked proteins, many of which are involved in adhesion), these may excite the initial formation of a microdomain. Once such a site has formed, avidity interactions will allow the recruitment of other proteins which prefer to be associated with such membranes, allowing maintenance and growth of such a site. Integrin ligation also stimulates intracellular signalling cascades which may also indirectly promote the local accumulations of microdomain-associated lipids. Due to their tight linkage to adhesion sites, these microdomains are presumably necessary for focal adhesion formation and function. Signalling through ligated integrins allows the recruitment of a large number of cytoplasmic proteins, which form the growing focal complex/adhesion and eventually, after Actin stress fibre formation in a RhoA-dependent manner, a mature focal adhesion is formed. At this point the plasma membrane of the mature adhesion is enriched in microdomain-associated components, and integrins form an indirect but strong connection between the extracellular matrix and the Actin cytoskeleton. Focal adhesions are very dynamic structures and are continuously remodelled, presumably in response to extracellular cues and the local availability of appropriate matrix components. Likely, some of this remodelling (or active maintenance) may occur locally though, for example, phosphorylation of cytoplasmic components of the adhesion site and the downstream effects of these events on the link between integrins and Actin. CLIC/GEEC endocytic tubules arise from mature adhesions which are known to have a relatively long lifetime, and thus this continual remodelling (as opposed to active disassembly) might also be provided by constitutive endocytosis from these sites. The lipids enriched in these adhesion sites include those most associated with Clathrin independent endocytosis. Therefore these sites most likely provide endocytic portals from which Clathrin-independent endocytic events preferentially originate, as has been observed for CLIC/GEEC endocytosis. Such constitutive endocytosis would likely have effects on the nature of the adhesion proteins themselves through modulation of their plasma membrane environments. It may also internalise transmembrane, or membrane-associated adhesion proteins themselves necessitating further recruitment of these proteins into the adhesion site (and their subsequent ligation) in order for maintenance of this site to occur. Focal adhesion disassembly (active disassembly) occurs in a piecemeal manner, consistent with gradual loss of components though an endocytic pathway. During the process of active adhesion site disassembly, a large portion of ordered plasma membrane is lost, presumably through the internalisation of the microdomains associated with these adhesion sites. This likely occurs through the endocytosis of adhesion-site associated lipids via the CLIC/GEEC pathway. During active disassembly, larger scale endocytosis ensues that is of a much greater magnitude than that which occurs constitutively. More adhesion site proteins will be internalised via this pathway than during the phase of active maintenance, including those that have stimulated adhesion site formation (e.g. integrins). The signals for the recruitment of cytoplasmic adhesion associated proteins into the site are therefore lost and adhesion markers are eventually no longer found at these sites. The plasma membrane at this disappearing site becomes disordered, concomitant with loss of adhesion to the surrounding matrix. Active adhesion disassembly may require concomitant inhibition of adhesion reformation and this may be provided by the local loss of adhesion proteins, or by changes in small G protein balance at these sites. Since Rho Kinase is required for the maintenance of focal adhesions and inhibits their active disassembly as well as endocytosis through the CLIC/GEEC pathway, this kinase likely needs to be locally inactivated in order for active disassembly by endocytic means to occur. Rho Kinase is activated by GIP-bound RhoA and stimulation of RhoA hydrolysis of GTP at this site by, for example, GRAF1 may allow this to occur locally. Indeed, the GAP domain of GRAF1 is required for its function. Coupled with this, PIX and PAK2, which favour Cdc42 and Rac1 activity, will change the local small G-protein balance in favour of these proteins at disassembling adhesion sites.

### CLIC/GEEC Versus Clathrin-Mediated Endocytosis

The model presented above suggests that the CLIC/GEEC endocytic pathway is primarily a lipid trafficking pathway, with proteins only being internalised via this pathway by virtue of their association with specific plasma membrane lipids. Indeed, aside from extracellular fluid, the protein cargoes identified for this pathway are microdomain associated. A specific lipid trafficking pathway contrasts starkly with Clathrin-mediated endocytic routes, which import cargoes that have been clustered by the endocytic machinery and which are likely pathways primarily for the specific trafficking of proteins themselves. Clathrin-mediated endocytosis occurs though the formation of spherical vesicles of a roughly constant diameter and appears to occur only when a sufficient number of specific cargo molecules have clustered in Clathrin-coated pits. Furthermore, many cargoes that enter via this pathway appear to signal primarily from the endosomal compartment. Perhaps this allows the regulation of signalling though a timer-like mechanism. Receptor ligation would activate the receptor which can then be recruited into a Clathrin-coated pit. This stage contributes little to intracellular signalling from the receptor. Once a specific number of activated receptors have accumulated in a CCP, the endocytic machinery induces its invagination and scission from the membrane to form a Clathrin-coated vesicle. Signalling can now proceed in concert with trafficking of this vesicle. Signalling will continue until this receptor is recycled to the plasma membrane, delivered to a lysosomal compartment for degradation, or acidified (in endosomal compartments) to allow dissociation of its ligand. The time between endocytosis (where productive signalling begins) and such an event (where signalling ends) would likely be roughly constant for each vesicle type. Trafficking occurs in, a cargo-driven manner, allowing versatility and specificity of the process. According to this novel model, a specific number of activated receptors would therefore deliver a single quantum of information to the cell through signalling cascades. Such a model would explain how signalling is efficiently regulated through endocytic routes, and how information processing to the interior of the cell about the nature of the extracellular milieu may occur. At the other end of the spectrum, the CLIC/GEEC endocytic pathway is presented not as a direct signalling platform for proteins, but as a method of lipid homeostasis that has knock-on effects that allow information processing. Likely, this pathway has no means by which to specifically duster protein cargoes other than via their preferential affinities for liquid-ordered (microdomain) over liquid-disordered regions of the plasma membrane. This pathway proceeds through the permissive nature of liquid-ordered regions of the plasma membrane for this type of internalisation event. This is not to say that it is not precisely regulated and coordinated but rather that it is the nature of these regions themselves that direct the endocytic process when stimulated to occur. It is unknown whether signalling can occur from proteins found in endosomal compartments of the CLIC/GEEC pathway. It has recently been shown that the oncoprotein ErbB2 (the HER2 epidermal growth factor receptor family protein) is found in CLIC/GEEC endocytic membranes and this finding may allow such issues to be directly addressed, as might the identification of other cargoes with cytoplasmic signalling domains. If a method for the intact biochemical isolation of CLIC/GEEC endocytic membranes can be established (although this is unlikely given their fragility), proteomic approaches may reveal such novel cargoes. Aside from inhibition of RhoA signalling through its effector kinase, it is unknown how the CLIC/GEEC pathway might be specifically activated. Since permissive lipids for this pathway are clustered in sites of adhesion, it is possible that intracellular signalling proteins can directly influence these sites, and these may affect the microdomains themselves, or the recruitment of proteins that are necessary for this pathway to proceed, such as small G-proteins or GRAF1. Being permissive for signalling, Clathrin-mediated endocytosis itself may therefore act upstream of the CLIC/GEEC pathway, but inhibition of this pathway still allows Clathrin-independent internalisation so remains an unlikely mechanism for Clathrin-independent endocytic regulation. Further studies will address these connections. Studies on Shiga Toxin uptake reported here have provided evidence that CLIC/GEEC and Clathrin-mediated endocytic routes direct cargoes to distinct locations within the cell and these are therefore not redundant pathways. However, Clathrin-mediated endocytosis can compensate for the CLIC/GEEC pathway in the internalisation of Shiga Toxin suggesting that there exists overlap of cargoes between these pathways, consistent with the observations that other CLIC/GEEC endocytic cargoes can ordinarily be internalised by both routes. Whether or not a cargo protein enters via the CLIC/GEEC or Clathrin-mediated endocytic pathway is likely dependent on its preference for different phases of the plasma membrane as well as the ability of the protein to interact directly with the Clathrin-mediated endocytic machinery.

### GRAF1 and Cell Migration

The role of membrane trafficking in cell migration has been hotly debated. The most compelling evidence for an important role came from the development and analysis of temperature-sensitive mutants of Dictyostelium discoideum, where the function of Nethylmaleimide sensitive factor (NSF; necessary for the disassembly of SNARE complexes once fusion of membranes has occurred and therefore necessary for membrane trafficking) can be turned on/off acutely by changing temperature. These cells are still capable of responding to chemotactic stimuli in the absence of NSF function, and produce leading edges towards these stimuli (which occurs in an Act independent manner). However, these cells do not move further towards these stimuli strongly suggesting that membrane trafficking is required for progression to frank, migration. It has also been shown that the direction of intracellular membrane flow is ordinarily towards the direction of the stimulus (since the plasma membrane was shown to move in the opposite direction) and that this is often accompanied by large changes in the surface area of cells. The largest body of evidence in this field supports a role for the endocytosis of adhesion receptors (likely from the rear of a migratory cell) and their eventual recycling to the membrane to take part in further adhesion events (likely at the leading edge where the membrane is coming into direct contact with matrix). It is, however, unknown whether this recycling occurs through the same pathways that supply the leading edge with the membranes that allow it to grow. The roles of small G-proteins in effecting the cytoskeletal changes required for cell migration have been the subject of intense study. The formation of large amounts of FActin is required for the formation of the leading edge of cells and signalling from ligated receptors at this site directs the formation of these filaments through Rho family small G proteins and their Actin-nucleating effectors. The molecular tranducers of these signals, and the mechanisms by which these operate, are well-understood. However, how such processes cooperate with membrane trafficking is unknown. Since there appears to be net membrane traffic towards the leading edge of cells, there must occur polarised exocytosis to this site, an inhibition of endocytosis from this site, or both.

There exists polarisation of active Rho family small G-proteins in migrating cells, and these proteins have been implicated directly in, or shown to be permissive for, endocytic events as discussed previously. Polarisation of the activity of these may allow polarised endocytosis to occur. The mechanisms by which polarised export might occur are unknown, but it is clear that this can certainly be managed, since synapses are regions well-known to be highly-regulated sites of polarised exocytosis. This might also be regulated by regional variations in small G-protein balance. Clathrin-mediated endocytosis is enriched towards the leading edges of cells so is unlikely to play a role in providing the required membrane redistribution, but probably allows the appropriate transduction of chemotactic stimuli. This is likely why CME is an essential component in cell migration regulation. Despite many unknowns, it is becoming clear that cell migration requires the intricate coordination of membrane trafficking, adhesion turnover and small G-protein regulation. The position of GRAF1 in its interactome places the protein in an ideal biochemical/network biological position to coordinate all three of these processes. Furthermore, studies presented herein have shown that GRAF1 is necessary for cell migration, endocytosis and focal adhesion disassembly, suggesting further that these processes are directly linked. Focal adhesions are disassembled at the rear of migrating cells, and new adhesions form at the leading edge to which membranes are trafficked. Endocytic disassembly of focal adhesions by GRAF1 and the CLIC/GEEC endocytic pathway from the rear of cells, with polarised trafficking of these membranes (and associated adhesion receptors) to their leading edge would provide an elegant mechanism by which cell migration might proceed. This is likely not produced by direct trafficking of membranes from the cell rear to the front, and the cell may use the Golgi apparatus (the likely destination for CLIC/GEEC pathway membranes) as an intermediate compartment in which sorting might occur. Integrins have been shown to be capable of being internalised by Clathrin-independent mechanisms and this study suggests that they enter via the CLIC/GEEC pathway. Furthermore, integrins are thought to recycle from the rear of migrating cells, through the Golgi apparatus, to the leading edge. Polarisation of membrane trafficking and adhesion receptor cycling is likely orchestrated by small G protein signalling in concert with cytosketal changes. The interrelationships of these processes are highly complex and difficult to study experimentally (since interference with any of these will have knock-on effects on the other). It is likely that systems biology and informatic approaches will be required to provide suitable models on which further predictive experiments can be based. The cell types used in the studies presented herein are relatively unpolarised cells (in which membrane trafficking is easiest to characterise given the large literature derived from study of such cells) and study of the CLIC/GEEC pathway in more polarised cells and cells that can undergo chemotaxis may also help to answer some of these outstanding questions. Preliminary experiments examining this pathway in leukocyte migration are underway.

### GRAF1 Family Members

GRAF1 is part of a wider subfamily of BAR domain-containing proteins that includes GRAF2, OPHN1 and the novel GRAF3. At least one member of this family has been conserved from fly to human suggesting that the family plays important roles in metazoa. The divergence from a single member of this family in flies to at least 4 in human suggests that gene duplication events resulted in several genes for this family which were individually selected for different functions throughout evolution. The close relationship of domain structure and ‘conserved’ residues in family members between species strongly suggests that this family has evolved by divergent rather than by convergent evolutionary mechanisms. GRAF1 and GRAF2 are more closely related to each other than to OPHN1. The studies presented here have shown that these proteins likely have similar functions in vivo, presumably in distinct cell types (although this remains to be established). Certainly, GRAF2 does not compensate for the loss of GRAF1 in the cell types in which this loss has been studied. However, the SH3 domains of GRAF1 and GRAF2 both bind Dynamin and FAIL, strongly suggesting that they regulate similar endocytic routes. The precise relationships between these proteins, and the novel GRAF3 proteins identified here, will be the subject of further study. While GRAF1 is expressed widely, it is brain-enriched suggesting that the process that it is required to regulate is most common in cells of this organ system. Astrocytes are highly migratory cells, suggesting why GRAF1 is found on adundant tubular compartments in these cells, and astrocytic migration is important for the maintenance of neuronal network function. OPHN1 is also a brain-enriched protein but is predominantly found in neurons where it is essential for dendritic spine morphogenesis278 and thereby likely plays a role in synaptic plasticity. It has been suggested that this function is provided by the RhoGAP activity of the protein. The studies herein strongly suggest that OPHN1 is a membrane trafficking protein, binding to and stabilising membranes similar to those stabilised by GRAF1 in the CLIC/GEEC endocytic pathway. Indeed, in order to dendritic spines to grow, it is likely that membranes must be trafficked to these sites in an analogous manner to those trafficking processes that allow cell migration to proceed. When overexpressed, GRAF1 and OPHN1 lipid binding domains bind to similar membranes in vivo. Furthermore, GRAF1 overpression in neurons results in extensive growth of dendritic arborisations. These findings suggest that these proteins might ordinarily perform similar functions in vivo, albeit in distinct cell types. Interestingly, the GIT1/PIX complex has also been shown to be essential for dendritic spine morphology and other events requiring cytoskeletal remodelling. However, since OPHN1 lacks the SH3 domain present in other family members, this makes generalisation difficult and the precise contribution of OPHN1 to membrane trafficking in neurons therefore requires direct experimental interrogation.

### GRAF1 Family Members and Disease

While it is clear that there are many similarities between GRAF family members, it is clear that they cannot (at least fully) compensate for each other when the expression of one member is lost or reduced. Mutations in OPHN1 are frequently found in human patients with X-linked mental retardation. While OPHN1-associated mental retardation was previously been thought to be a non-syndromic condition, it has now been shown that the brains of these patients have a variety of associated clinical and structural abnormalities. By MRI, specific patterns of cerebellar dysgenesis and atrophy of cortico-subcortical fibres have been found in sufferers. These patients become hypotonic after birth and have a delay in motor development, as well as cerebellar signs and moderate to severe mental retardation. How these signs relate to the requirement for this protein in appropriate dendritic spine morphogenesis is unknown, but neurons that do not make appropriate connections are known to undergo apoptosis during development, and perhaps this contributes at least to the hypoplasia and atrophy in the brains of these patients. GRAF1 is expressed in cells of lymphoid origin, where deletions, truncations or translocations of one GRAF1 allele have been found in parallel with mutations of the other allele in patients with Acute Myeloid Leukaemia and Myelodysplastic syndrome. The nature of these mutations include those predicted to inhibit the function of the GAP domain of GRAF1, as well as frameshifts that likely result in truncated proteins similar to the dominant-negative protein characterised in the studies herein. Moreover, ˜38% percent of biopsies of bone marrow from patients with Acute Myeloid Leukaemia or Myelodysplastic Syndrome, exhibit GRAF1 promoter methylation which is associated with reduced protein expression. Ordinarily this site is unmethylated. Acute Myeloid Leukaemia is characterised by the increased proliferation of pathogenic ‘blast cells’ which retain proliferative capacities and do not appropriately differentiate, much like the endogenous behaviour of tissue stem cells. Loss of GRAF1 expression has no observable effect on cell proliferation in vitro so it is unlikely that this protein has a direct role in cell cycle regulation. Interestingly, it has recently been shown that intraperitoneal injection of monoclonal antibodies directed against CD44, which is found massively upregulated on the surface of the pathogenic blast cells, can eradicate a mouse model of the disease (where human Acute Myeloid Leukaemic cells are transplanted into host mice). This likely comes from the inability of CD44-inhibited cells to find a peripheral niche (in which residence is required) in order for proliferation to occur. Increased expression of a protein on the surface of a cell can result from either increased expression, or from a defect in endocytosis of that protein. The results presented here suggest that a reduction in GRAF1 expression might promote leukaemogenesis by inhibiting the function of the CLIC/GEEC endocytic pathway. This would lead to the increased surface expression of proteins which result in increased cellular adhesion, a process required for blast cells to bind to, and remain in a suitable niche in which proliferation may occur. In this model, loss of GRAF1 expression would promote leukaemogenesis, but may require initiating mutations such as increased expression of oncogenes that might increase the capacities of cells to proliferate, or inhibit their capacities to differentiate. If the above model is true, it suggests a potential (and inexpensive) therapeutic approach for a subset of patients with Acute Myeloid Leukaemia in whom GRAF1 function and expression is predicted to be normal, or patients in whom GRAF1 function is predicted to be normal but expression is reduced through promoter methylation. It has been shown that inhibition of the major RhoA effector Rho Kinase can upregulate the CLIC/GEEC endocytic pathway (which is predicted to be deficient in the pathogenic cells from these patients). Treatment of patients with such an inhibitor might therefore increase the internalisation of adhesion receptors on their pathogenic cells, thereby inhibiting the adhesion step(s) required for homing of these cells to their niches. Such an inhibitor, fasudil, has recently been successfully tested in clinical trials for acute ischaemic stroke and was shown to be non-toxic. This inhibitor will be tested in mouse models of Acute Myeloid Leukaemia in future studies. It is unlikely to be of any benefit in patients with GRAF1 mutations that are predicted to act as dominant-negatives, since normal GRAF1 function would be required for this approach to be successful. Tumour cells acquire migratory phenotypes that are necessary for their invasion into surrounding tissues and metastasis. Therefore at first glance the role of GRAF1 as a tumour suppressor in haematopoietic cells appears antithetical given its pro-migratory roles. However, this must be considered in the context of the important differences between bone marrow-derived cancers, and their solid organ counterparts. In the bone marrow, cells are continually released into the circulation as part of the normal replenishment of blood cells that have been broken down in remote sites such as the spleen. This cycle of birth and release is presumably why single mutations in precursor cells in the bone marrow that increase proliferation are often sufficient to be pathogenic. This is in stark contrast to the progression of solid organ malignancies which must overcome many barriers in order to metastasise. Solid organ malignancies acquire mutations that enhance their proliferation, inhibit anti-proliferative signals, inhibit apoptotic signals, and induce the formation of new blood vessels to deliver nutrients and remove metabolic waste products. Later they accrue mutations in order to become migratory and invade the surrounding tissue and the basement membrane, before metastasis can occur through their migration into lymph or blood vessels, subsequent adhesion to a target site, and extravasation into this tissue. This is de facto a much more complicated process than the series of events that occur during leukaemogenesis, and requires many mutations to occur in a coordinated fashion. This suggests that although GRAF1 acts as a tumour suppressor in white blood cells, it might also act as an oncogene during migration and invasion phases of malignant progression of solid tumours (since GRAF1 positively regulates, and is required for, cell migration). This remains to be intensively studied but a very recent study has reported that GRAF1 expression is upregulated in brain metastases from primary lung adenocarcomas. Further, OPHN1 has been shown to be upregulated in colinic adenocarcinomas and invading gastric carcinomas. Whether mutations in GRAF2 and GRAF3 are also linked to human disease remains to be investigated.

We disclose novel methods by which to experimentally manipulate the CLIC/GEEC pathway.

### BRIEF DESCRIPTION OF THE DRAWINGS

FIG. 1 GRAF1 localises to a prevalent tubular endocytic pathway capable of large amounts of membrane redistribution, a, Domain architecture of GRAF1 and the sites of introduced functional mutations (*). b, Different forms of GRAF1 in adult rat brain and their differential presence/absence in cultured SH-SY5Y (human neuroblastoma), HeLa (human fibroblast), K562 (human Chronic Myeloid Leukaemia), and MEF (mouse embryonic fibroblast), cells as detected by immunoblotting using a polyclonal antibody directed against the PH and GAP domains. c, Confocal micrographs showing a tubular and punctate localisation of endogenous GRAF1 in primary astrocytes and HeLa cells. d, GRAF1-positive tubules are derived from the plasma membrane as shown by co-labelling with the membrane dye DiI after 5 minutes. These structures are extensively co-labelled with internalised dextran after 1 and 5 minutes. e, Immunoblot demonstrating that siRNA treatment efficiently reduces the expression of GRAF1. f, Cells depleted of GRAF1 show a major reduction in fluid phase endocytosis as shown by the decrease in uptake of FITC-labelled dextran, (control siRNA (n=5), siRNAa (n=8) or AP2 siRNA (n=3)). The error bars show the standard error of the mean. g, Epifluorescent micrographs of HeLa cells transfected with a control siRNA, or siRNAa, and then incubated with dextran. Note the profound reduction in tubular dextran uptake observed in GRAF1-depleted cells.

FIG. 2 GRAF1 interacts with highly-curved, PtdIns(4,5)P2-enriched membranes and components of focal complex/adhesion disassembly machinery, a, b, The N-terminal BAR and PH domains constitute a membrane binding region of GRAF1 that shows a preference for binding to smaller-sized liposomes and liposomes containing the phosphoinositide PtdIns(4,5)P2 in liposome co-sedimentation assays. The error bars in (a) show 95% confidence intervals (calculated by t-tests) for each condition. c, Immunoprecipitation of GRAF1 from rat brain cytosol reveals a GRAF1/dynamin1/GIT1 complex as identified by mass spectrometry and confirmed by Western blot. d, The SH3 domain of GRAM binds dynamin, FAK, caskin1, and synaptojanin in a pull-down assay from rat brain cytosol. (Lower right) Immunoblot of GRAM after immunoprecipitation of pFAK from rat brain cytosol. e, Schematic representation of the GRAF1 interactome, showing the interactions that link focal adhesion turnover, small G-protein regulation and GRAF1-mediated membrane trafficking into a machinery for cell migration. Dotted lines shown interactions known to be directly activating/inhibiting the function of another depicted protein. f, Overexpression of GRAF1 in HeLa cells induces a profound morphological change coincident with the downregulation of focal adhesions, shown by the loss of the typical vinculin stain at the periphery of overexpressing cells.

FIG. 3 GRAF1 is required for focal complex/adhesion turnover and for cellular migration. a, Epifluorescent micrographs of endogenous GRAF1 (left panel) or overexpressed myctagged GRAF1 BAR+PH proteins (right panel) in HeLa cells co-stained for paxillin. Note that the peripheral ends of GRAF1-positive tubes are at sites of focal adhesion (arrow heads). b, GRAF1 punctae co-localise with vinculin at focal complexes (arrow heads) but not at mature focal adhesions. c, Overexpression of the GTPase activation deficient mutant of GRAF1 (GFP-GRAF1 R412D) results in a large increase in the amount of colocalisation of vinculin and GRAF1 at focal complexes. d, Induction of focal adhesion turnover in HeLa cells by the ROCK inhibitor Y-27632 increases the number of GRAF1-positive tubules and the localisation of GRAF1 to focal complexes where it co-localises with clusters of 1-integrin (see merge of boxed area). e, Depletion of GRAM expression in HeLa cells results in a profound increase in the number of vinculin-positive subnuclear focal adhesions. f, Quantification of cells from (e) (n=78 for control siRNA-treated cells, n=91 for GRAF1 siRNA-treated cells). g, Principle of electrically ᵢ®induced and ᵢ®monitored wound healing assay. Graph showing the recovery from electrical wound healing of a confluent HeLa cell layer, the cells in which were previously transfected with a control siRNA or siRNAa. Shaded areas represent one standard deviation above and below the mean values for each condition.

FIG. 4 shows a diagram of evolution of GRAF and OPHN1 family members. In more detail, it shows a phylogenetic tree depicting predicted (relatively-scaled) evolutionary distances for GRAF1- and OPHN-like sequences (ie. GRAF protein family members) from a range of species. Only the sequences' species of origin are shown. Protein nodes in the same group are depicted in the same colour. Note the 3 main GRAF families present in higher eukaryotes, and the presence of OPHN family sequences as a fourth family of GRAF paralogues present in vertebrate lineages. For sequences that do not fall obviously into the GRAF1/GRAF2/GRAF3/OPHN protein families, the presence or absence of a predicted SH3 domain in these sequences is noted.

FIG. 5 shows a diagram of evolution of GRAF and OPHN1 family members. In more detail, it shows a phylogenetic tree depicting predicted (relatively-scaled) evolutionary distances for GRAF1- and OPHN-like sequences from a range of species as shown in FIG. 4. The sequences' accession numbers are shown here.

FIG. 6 Model of GRAF1- and clathrin-dependent endocytic mechanisms. a, Schematic model depicting the parallel nature of GRAF1- and clathrin-dependent endocytic mechanisms highlighting the different endocytic proteins involved in tubular versus vesicular endocytic mechanisms. The lower panel depicts the local anatomy of a disassembling focal adhesion occurring in a small G-protein and GRAF-dependent manner. For simplicity cytoskeletal elements are omitted.

FIG. 7 GRAF1 mediated trafficking is distinct from clathrin dependent endocytosis. a-d, Confocal micrographs of untransfected HeLa cells, or HeLa cells overexpressing myc-tagged GRAF1 as indicated and co-stained for clathrin (a), transferrin receptor (b), transferrin endocytosed at 37 degrees for 10 minutes (c), or CTxB endocytosed for 5 minutes at 37 degrees (d). Note that neither endogenous nor overexpressed GRAF1 colocalises with clathrin-dependent endocytic markers, but that CTxB is found in GRAF1-positive tubules. e, Confocal micrograph of HeLa cells treated with a control siRNA or siRNA against GRAF1 before incubation with transferrin for 15 minutes at 37 degrees. Note that transferrin uptake is unaltered in GRAF1 depleted cells.

FIG. 8 GRAF1 mediated trafficking is dependent upon dynamin and small G-proteins. a, HeLa cells incubated either with medium including vehicle (DMSO) or 50 um dynasore for 15 minutes before addition of dextran to these, cells for a further 15 minutes, (with DMSO/dynasore concentrations being retained throughout), before fixation and staining for GRAF1, dextran and paxillin. Note the reduction in GRAF1-positive tubular structures and a redistribution of GRAF1 to the cellular periphery (where it is predominantly located in punctate and ring structures on the basal surface of cells) in dynasore-treated cells. Note also the profound reduction in tubular dextran uptake in these cells. b, Epifluorescent micrograph of endogenous GRAF1 in HeLa cells transfected with HA tagged RhoA N19 or GFP-tagged cdc42Q61L. Note the absence of GRAF1-positive tubules in transfected cells. Scale bars=10 um.

FIG. 9 Membrane tubules generated by the GRAF1 BAR+PH domain in vivo are mobile structures dependent upon microtubules. a, Representative Coomassie-stained gel of a typical liposome co-sedimentation assay with GST-tagged BAR+PH and liposomes of different diameters quantified in FIG. 2a (P=pellet, S=supernatant). b, Confocal micrographs showing the tubular localisation of overexpressed myc-tagged BAR+PH protein in HeLa cells and the cytoplasmic localisation of a similarly overexpressed protein with a BAR domain mutation (KK131/132EE). Scale bars=10 um. c, Electron micrographs of negatively-stained liposomes incubated in the presence or absence of GST-tagged GRAF1 BAR+PH protein. Note the presence of tubular structures present with BAR+PH protein and their absence in the control incubation. Scale bars=200 nm. d, Confocal micrographs of cells overexpressing GFP-tagged GRAF1 BAR+PH protein left untreated (Panel 1) or treated with 1 uM nocodazole for 1 hr and fixed immediately (Panel 2) or left to recover for 30 mins after nocodazole washout (Panel 3). Cells were then stained for beta-tubulin (lower images). GRAF1 BAR+PH-positive tubular structures were not present in nocodazole treated cells but recovered after nocodazole washout where they are observed to colocalise with beta-tubulin. e, Live cell spinning disc confocal imaging of a GFP-tagged GRAF1 BAR+PHover expressing cell at 20° C. These images are taken 37 seconds apart and yellow structures in the merged image show elements which have not moved in this time, green and red elements show elements which have moved (green/red), appeared (green), or disappeared (red). Some of these movements are tracked in the overlay. The full time course of this experiment can be seen in Supplementary Movie 1.

FIG. 10 GRAF1 binds directly to dynamin1 and dynamin2. a, Coomassie-stained gel and confirmatory Western blots of co-immunoprecipitation experiments in rat brain cytosol (cyt) performed with either control pre-immunisation serum (pre-serum) or Ab2 (towards GRAF1 SH3 domain). b, Coomassie-stained gel and Western blots of pull-down experiments in HeLa cell cytosol with beads bound to GST (control) or GST-tagged GRAF1 SH3 domain. Dynamin2 was identified by mass spectrometry as described. c, Coomassie stained gel of pull-down experiments from rat brain cytosol with GST-tagged GRAF1/GRAF2 SH3 domains, GST-tagged amphiphysin2 (Amph) SH3 domain or GST alone. Note that both GRAF1 and GRAF2 bind caskin1, FAK and dynamin. d, In vitro pull down experiment with beads coupled to equimolar amounts of GST, GSTGRAF1 SH3, Amphiphysin SH3, and GRAF1 BAR+PH proteins incubated with soluble purified dynamin. Pellet fractions represent dynamin bound to the protein of interest.

FIG. 11 GRAF1 produces a profound morphological change in cells in a GAP domain-dependent manner. Tubular endocytosis occurs from focal adhesions. a, Confocal micrographs of endogenous GRAF1 (left panel) or overexpressed myctagged GRAF1 (right panel) in HeLa cells showing a similar tubular and punctate stain. b, High overexpression levels of myc-tagged GRAF1 but not the GTPase activation deficient myc-tagged GRAF1 (R412D) results in a collapse of cell morphology. c, Epifluorescent micrograph of GRAF1 and vinculin localisation in HeLa cells after 10 minutes of dextran uptake at 37 degrees. Note the colocalisation of ending GRAF1- and dextran-positive tubes and their peripheral colocalisation with focal adhesions.

FIG. 12 Induction of focal adhesion turnover profoundly increases the number of GRAF1-positive tubules and recruits GRAF1 and dynamin to disassembling focal complexes. a-c, Epifluorescent micrographs of HeLa cells treated with 40 uM of the ROCK inhibitor Y-27632 for 40 minutes prior to fixation and immunofluorescent staining using antibodies against indicated proteins. Note that endogenous GRAF1 tubes originate from within rings of beta1-integrins (a). Note that after treatment with Y-27632, vinculin is found mostly in focal complexes where it co-localises with GRAF1 (b), and that Dynamin similarly colocalises with GRAF1 (c).

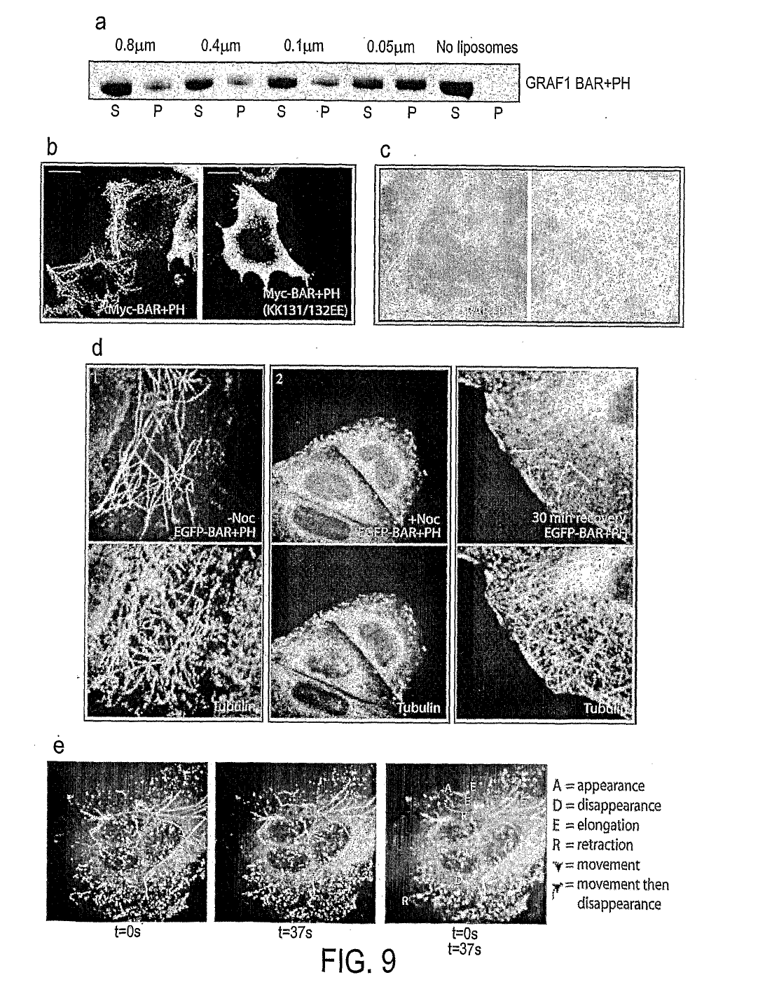

Supplementary Movie 1 GRAF1 BAR+PH localised to motile tubular/vesicular structures. This is the full time series of the experiments described in FIG. 9e. Experiment was performed at 20° C. Playback speed is 40 times faster than acquisition.

Supplementary Movie 2 GRAF1 BAR+PH positive tubules are capable of extension and retraction. When motile they move at speeds of around 0.2-0.3 um/sec at 20° C.

Supplementary Movie 3 GRAF1 BAR+PH positive tubules are capable of high speed trafficking. Speeds of up to 0.6 um/sec were observed at 20° C.

The invention is now described by way of example. These examples are intended to be illustrative, and are not intended to limit the appended claims.

### EXAMPLES

### Overview

Cell migration requires the intricate coordination of membrane and protein redistribution, changes in cytoskeletal architecture, and focal complex/adhesion turnover. However, the mechanisms by which this coordination occurs are unclear. The importance of elucidating the cell biological mechanisms of migration is underlined by the fundamental roles this process plays in tissue development, immunology, neurobiology, and the invasion and metastasis phases of oncogenesis. Members of the Rho family of small G proteins have been shown to be master regulators of cell migration. Here it is shown that the Rho GAP domain-containing protein GRAF1 defines and regulates a major Clathrin independent endocytic pathway responsible for the internalisation of bacterial exotoxins, GPI-linked proteins, and extracellular fluid. This endocytic pathway is independent of Clathrin, Caveolin and Flotillin, but can be further defined by the presence of Rab8. Since GRAF1 is a multidomain protein, biochemical dissection of this endocytic route could then be performed. GRAF1 localises to highly dynamic PtdIns(4,5)P2-enriched membranes via N-terminal BAR and PH domains, and interacts with proteins including Dynamin, GIT1, FAK, and PAK2. Since these latter proteins promote the disassembly of focal adhesions, this places GRAF1 in a position whereby it may coordinate cell migratory events through coupling membrane redistribution and focal adhesion turnover. Indeed, GRAM is necessary for turnover of focal complexes/adhesions, and GRAF1-dependent endocytosis occurs from these sites in a small G-protein dependent manner. Further, GRAM is necessary for cell migration. The studies presented thereby provide the first markers for this prevalent endocytic pathway, and reveal dynamic cellular anatomy responsible for the coupling of endocytosis and cell migration.

### Example 1

### The GTPase Regulator Associated with Focal Adhesion Kinase (GRAF) Family of Bar Domain-Containing Proteins

Using protein sequence homology searches in human databases it is possible to identify subsets of BAR domain-containing proteins based either upon homology within the predicted BAR domain itself, or from protein sequences from full predicted proteins. One such subset comprises Oligophrenin 1 (OPHN1), GTPase Regulator Associated with Focal Adhesion Kinase1 (GRAF1) and GRAF2. All three proteins comprise a predicted N-terminal BAR domain, followed by a PH domain, GAP domain and proline-rich region. GRAF1 and GRAF2 also contain a predicted C-terminal SH3 domain that is absent in OPHN1. This has been previously described as the Oligophrenin protein family but, since OPHN1 lacks this additional domain, this family is better described as the GRAF protein family. OPHN1 has been shown to be capable of enhancing GTPase hydrolysis of RhoA, Rac1 and Cdc42 in vitro and hence does not discriminate between these with high specificity (although it may do so on the basis of spatial localisation in vivo). The greatest enhancement of hydrolysis was observed for RhoA. Interestingly, while overexpression of full length OPHN1 in vivo resulted in mild increases in active Cdc42 levels, overexpression of the C-terminal region of the protein (including only the predicted GAP and proline-rich domain) resulted in almost undetectable levels of active RhoA, Cdc42 or Rac1. These results suggest that ordinarily the GAP domain of OPHN1 is negatively regulated by an N-terminal region of the protein which includes the BAR domain. Overexpressed OPHN1 in COS-7 cells was found to colocalise with F-Actin and this colocalisation was shown to require only the extreme C-terminus of the protein (at most the last 125 amino acids). Co-sedimentation analyses of purified OPHN1 C-terminus with F-Actin suggested that interaction of this region with the cytoskeleton may be direct. While overexpression of full length OPHN1 did not cause observable phenotypic changes in F-Actin distribution, the numbers of lamellopodia and filopodia were specifically reduced in GAP domain-overexpressing cells. This further suggests that the N-terminal region of OPHN1 negatively regulates its GAP activity. OPHN1 mRNA levels in adult brain have been shown to be highest in the olfactory bulb, cortex, hippocampal pyramidal cell layers, as well as granular cells of the dentate gyrus, and Purkinje cells of the cerebellum. Many of these regions show high degrees of synaptic plasticity. Expression was observed in both neuronal and glial cells, in myelin sheaths surrounding neurons in the parasympathetic and sensory-somatic nervous systems, as well as in chromaffin cells of the adrenal medulla and sympathetic ganglia and neurons of enteric neural plexuses. OPHN1 mRNA was found at higher levels in foetal than in adult brain and, using specific antibodies to OPHN1, protein levels were found to be similar for newborn and adult brains. siRNA treatment of cultured neurons to reduce OPHN1 levels resulted in a consistent reduction in the length of dendritic spines, consistent with a role for this protein in synaptic plasticity. Indeed, OPHN1 is found frequently mutated in X-linked mental retardation. However, the mechanism by which occurs is currently unclear. GRAF1 appears to act as a tumour suppressor in leukocytes, where deletions, truncations and mutations in both alleles have been found associated with Acute Myeloid Leukaemia and Myelodysplastic Syndrome. The G:C-rich promoter of GRAF1 is ordinarily unmethylated, but ˜38% percent of biopsies of bone marrow from patients with these conditions exhibit GRAF1 promoter methylation which is associated with reduced protein expression289. In order to understand the dysregulation of this protein in disease, it is essential to first elucidate its normal cell biological and physiological function(s). However, studies on GRAF1 have been limited to structural analyses and the identification of interacting proteins. The crystal structure of the GAP domain of GRAF1 has been solved, as has as a solution structure of the SH3 domain (PDB reference 1 UGV). These structures have, as yet, added little to our understanding of the biology of GRAF1. GRAF1 exhibits GAP activity for RhoA and Cdc42 in vitro, and favours the downregulation of RhoA activity in vivo. It has also been shown to interact with the kinases FAK and PKNβ. Interestingly, FAK depletion is known to reduce the amount of SV40 internalisation, which occurs via caveolae- or microdomain-dependent endocytosis. Furthermore, FAK is known to regulate focal adhesion turnover. Since GRAF1 has a BAR domain, it is likely to be involved in membrane trafficking, and may be involved in producing or stabilising the membrane deformation required for endocytic events. The studies presented herein provide greater clarity to the field of Clathrin-independent endocytosis which, as described, has been inextensively characterised. Endocytic pathways necessarily require the function of proteins that can influence membrane curvature directly, although no such proteins have been ascribed to Clathrin-independent endocytic pathways. Since BAR domains are involved in membrane sculpting events, and since there are a variety of BAR domain-containing proteins in mammalian proteomes, at least one of these proteins may play a role in Clathrin independent endocytic events. GRAF1 has a predicted regulatory domain for Rho family small G-proteins which have been implicated in Clathrin-independent endocytic events and GRAF1 may provide a link between membrane deformation and the activity of these proteins. The BAR domain-containing protein GRAF1 binds Focal Adhesion Kinase which has previously been shown to be necessary for certain Clathrin-independent endocytic events. GRAF1 might therefore play a role in Clathrin-independent endocytosis. If GRAF1, or another BAR domain-containing protein, can be ascribed to a Clathrin independent endocytic pathway, analysis of its biochemistry and cell biology may provide further markers of, and mechanistic insight into, Clathrin-independent endocytosis. No cell biological or physiological functions have been definitely ascribed to any Clathrin-independent endocytic pathway before this study. Thorough biochemical and cell biological analysis of definitive markers of Clathrin-independent endocytic pathways reveals their functional relevance. Study of the normal cell biological functions of GRAF family members provides insight into how their functional loss or hyperactivity may contribute to disease processes to which such dysregulation is linked.

### Example 2

Characterisation of GRAF family members In silico characterisation of the GRAF family of putative BAR domain containing proteins: GRAF1 is a putative BAR domain-containing multidomain protein A BAR sequence alignment was produced from overlaying the previously-solved Drosophila melanogaster Amphiphysin and Arfaptin2 BAR domain structures and was used in repeated iterations of PSI-BLAST (http://ncbi.nlm.nih.gov/BLAST) to identify regions of predicted human protein sequences which may contain BAR domains. These regions were then verified by ClustalW alignment (with an ‘open gap penalty’ of 100, an ‘extend gap penalty’ of 0.5, and a ‘delay divergence setting’ of 40%) and checked for predicted a-helical content using PredictProtein (http://www.predictprotein.org) since secondary structure in all published crystal structures of BAR domains is of this type. They were also compared with sequences encoding F-BAR/IMD domains to remove sequences predicted to encode these coiled coil components; these regions were invariably of lower homology to the input BAR sequence. Protein sequences identified by these techniques were then cross-checked with the conserved domain database to identify other regions of interest. Such techniques were capable of identifying a diverse range of proteins with known and putative BAR domains. One member from each of the protein, families identified, with its predicted domain organization is shown. A subset of the domains identified had a predicted N-terminal amphipathic helix. Such proteins have previously been classified as N-BAR domain-containing proteins. Many BAR and N-BAR domain-containing proteins are large multi domain-containing proteins, with some including predicted GTPase Activating Protein (GAP) and Guanine nucleotide Exchange Factor (GEF) domains which are known to be involved in the regulation of small G-proteins of the Arf and Rho families. Others are also predicted to contain protein-protein interaction regions such as Src Homology 3 (SH3) domains, PSD95/DlgA/Zo1 (PDZ) domains, or ankyrin repeats. In terms of predicted domain organisation, GTPase Regulator Associated with Focal adhesion kinase1 (GRAF1), GRAF2 and Oligophrenin1 (OPHN1) are members of one subfamily of these predicted human proteins. Each of these proteins are predicted to comprise an N-terminal BAR domain, with subsequent PH, RhoGAP and proline-rich domains. GRAF1 and GRAF2 are also predicted to have a C-terminal SH3 domain. An alignment of the full length sequences of these proteins is produced. GRAF1 and GRAF2 share ˜58% overall amino acid identify, while GRAF1 and OPHN1 share ˜45% overall identity. GRAF2 and OPHN1 share ˜44% overall identity.

At least one member of the GRAF protein family is conserved from fly to human pBlast searches were then performed using the full length GRAF1 sequence as bait in non-redundant sequence databases without restriction of organism. The sequences retrieved were checked manually to ensure that no potentially redundant sequences were included. Where it appeared that incomplete sequences, or different isoforms of the same protein sequence, had been retrieved, the longest sequence of each type was retained and the rest discarded. Proteins more similar in sequence to OPHN1 than GRAF1/GRAF2 were discarded from this analysis and treated separately. Retained sequences (an underestimation of the complete array of sequences due to the high stringency of the selection procedure and the incomplete nature of reference databases) were then subjected to Needleman-Wunsch global pairwise alignment in Geneious Pro 3.0.4 using a Blosum62 cost matrix, a ‘gap open penalty’ of 12, and a ‘gap extension penalty’ of 3. Aligned sequences were then used to build a phylogenetic tree using a Jukes Cantor genetic distance model with a neighbour-joining method. The same tree with accession number annotations for these protein sequences is produced. From this tree, 5 groups of GRAF1-related sequences were identified. An ancestral set of sequences (orange nodes) were identified in insects and worms, species in Predicted domain boundaries (from the conserved domain database) for GRAF1 are 20-220 (BAR domain), 268-367 (PH domain), 364-563 (GAP domain) and 705-757 (SH3 domain). The analysis is conducted using the following sequences: Rattus norvegicus Gallus gallus Gallus gallus Homo sapiens Tetraodon nigroviridis Danio rerio Canis lupus familiaris Canis lupus familiaris Xenopus laevis Mus musculus Homo sapiens Caenorhabditis elegans Rattus norvegicus Gallus gallus Macaca mulatta Macaca mulatta Homo sapiens Canis lupus familiaris Rattus norvegicus Mus musculus Monodelphis domestica Bos taurus Apis mellifera Tetraodon nigroviridis Pan troglodytes Mus musculus Xenopus laevis Drosophila melanogaster Monodelphis domestica Tetraodon nigroviridis Mus musculus Caenorhabditis briggsae Bos taurus (Accession numbers XP_{—}225989 XP_{—}417185 CAG30928 BAB61771 CAG11070 NP_{—}001038715 XP_{—}533968 XP_{—}535224 NP_{—}001086611 XP_{—}989830 AAH68555 NP_{—}741163 XP_{—}001065920 XP_{—}001232915 XP_{—}001096942 XP_{—}001091450 XP_{—}001127597 XP_{—}539757 XP_{—}576354 XP_{—}996933 XP_{—}001366867 NP_{—}001070298 XP_{—}001122822 CAG00508 XP_{—}518009 NP_{—}780373 NP_{—}001088562 NP_{—}573070 XP_{—}001365377 CAG11712 NP_{—}084389 CAE64342 XP_{—}618416) GRAF1-like sequences (blue nodes; these are significantly more similar to human GRAF1 than GRAF2) were found in species including pufferfish, frog, chicken, and mammals. GRAF2-like sequences (red nodes; which are significantly more similar to human GRAF2 than GRAF1) were also found in frogs, chicken, and mammals. Interestingly, a further group of sequences, which are significantly more similar to themselves than to either GRAF1 or GRAF2 (with each of which they share roughly equal similarity), were identified by these analyses (see green nodes). Such sequences can be found in the mammalian lineage as well as the chicken. These sequences are often confusingly annotated, e.g. the human XP_{—}001127597 which is annotated as ‘similar to Oligophrenin 1 isoform 2’ despite a predicted C-terminal SH3 domain that is not found in OPHN1 sequences and a greater sequence identity to GRAF1 and GRAF2 than to OPHN1. This novel family of proteins is therefore named here as a further GRAF subfamily: the GRAF3 family of proteins. These sequences share more similarity to themselves than ancestral/GRAF1/GRAF2/OPHN1 sequences and have therefore likely undergone some form of positive selection throughout evolution. A final group of sequences, which do not fit into GRAF1/GRAF2/GRAF3 families (but which are more similar to these than to OPHN1 protein sequences), are shown as dark grey nodes. These appear to represent divergent sequences and are found in fish species. Taken together, these results suggest that gene duplication events occurred in one or more common ancestral species of birds, mammals and fish, which was not shared with worms or insects. By contrast with GRAF protein sequences, only a maximum of one OPHN1-type protein sequence in each species was identified after similar analyses. Here the phylogenetic tree that was produced more closely resembled that produced from evolutionary genomic analyses. The presence of an OPHN1-like sequence in the yellow fever mosquito Aedes aegypti suggests that a gene duplication event (presumably of an ancestral sequence that was also eventually responsible for GRAF1/GRAF2/GRAF3 sequences) occurred in a common ancestor of mammals and mosquitoes. OPHN1-like sequences are not present in Drosophila melanogaster. Convergent evolution to OPHN1-like protein sequences cannot be ruled out, although the high similarity observed between OPHN1 and GRAF1 protein sequences in higher mammals suggests that this did not occur.

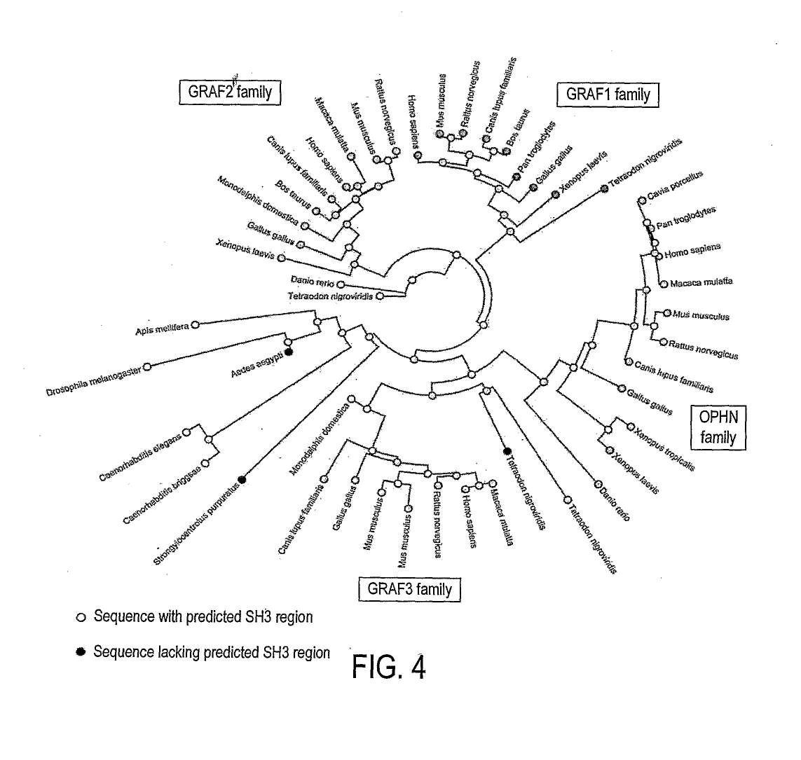

FIG. 4 and FIG. 5 show the phylogenetic trees (one with organisms and one with accession numbers) of the 4 GRAF paralogues.

### Identification of Conserved Residues in GRAF Family Proteins

There are a number of conserved residues that can be discerned in human GRAF1, GRAF2 and OPHN1. The GRAF1 family sequences (excluding the Tetraodon nigroviridis sequence which has no predicted SH3 domain and would therefore confound analysis of conserved sites in this domain) were then aligned to identify evolutionarily-conserved residues in family members. These sequences have a pairwise similarity of 80% and a sequence identity of 55%. Homology is greatest in the predicted BAR, PH and GAP domain sequences, with considerable divergence in the proline-rich sequences. To identify completely-conserved residues, alignments were performed with these sequences together with an ancestral sequence (from D. melanogaster). From this alignment plot, it can be seen that both dog (Canis lupus familiaris) and rat (Rattus norvegicus) sequences have significant N terminal extensions that are not present in the other species. This extension is 268 residues long in dog, and 100 residues long in rat. Using Globprot 2 to identify putative globular and disordered sequences, both of these N-terminal extensions were predicted to be largely disordered in both sequences so likely do not include ordered domains. These sequences share some identity, particularly in the stretch of residues from 149-173 in the dog sequence which are 92% identical to that of the rat. The dog N-terminal sequence is significantly more proline-rich than that of the rat, with 14.5% prolines, 16% arginines and, since it is predicted to be disordered, can therefore be considered a second prolinerich domain. The consensus sequence from this alignment was then extracted. This consensus sequence was then aligned with the human GRAF1 sequence to identify the residues in this sequence that have been absolutely conserved throughout its evolution. A triple lysine motif in the predicted BAR domain (residues 131-133 of the human sequence; highlighted in red box) is completely conserved and aligns with residues in D. melanogaster Amphiphysin BAR domain which are necessary for the electrostatic membrane binding of the dimeric Amphiphysin BAR module. A further conserved residue of interest is R412 (of the human sequence; highlighted in blue box). This aligns with an arginine in other RhoGAP domains known to act as a ‘finger’ in stimulating GTP hydrolysis by Rho family small G-proteins. It also is the likely catalytic residue in the GRAF1

GAP domain identified from analysis of its structure. The positively-charged side chain of this arginine inserts into the active site of the small G-protein, compensating the negative charges of the oxygen atoms of the γ-phosphate of ATP, thereby stabilising the transition rate of the hydrolysis reaction295. Both the triple lysine motif and arginine finger are also found in GRAF2 and OPHN1 protein sequences. Other absolutely conserved residues are also observed.

GRAF Family Members Bind Membranes In Vivo and In Vitro Through, their N Termini Antibodies Directed Against GRAF1 Domains Recognise a ˜94 KDa Protein Present in Rat Brain and a Variety of Cell Lines

There are two splice variants present in sequence databases for GRAM, one predicted to encode a protein of 759 amino acids (used for the alignments in the previous examples), while the other, more common sequence is predicted to encode a protein of 814 amino acids. The two versions differ only by the presence or absence of a stretch of 55 residues present at the C-terminal end of the proline-rich domain in the long form. Examination of this sequence by PredictProtein predicted the insert to be ˜50% a-helical and exist as a compact globular domain. cDNA fragments encoding the long version of GRAF1 full length (residues 1-814), GRAF1 PH+GAP (residues 267-576), and GRAM SH3 (residues 749-814) were cloned into pGEX-4-T2 vectors with 5′ GST tags. These proteins were expressed in E. Coli and purified using glutathione Sepharose beads and gel filtration. These proteins were then used to immunise rabbit and chicken hosts. Polyclonal antisera produced post immunisation were then depleted to remove antibodies recognising GST and then affinity-purified as described. Affinity purification was performed against purified proteins from a different purification procedure than was used to generate protein to immunise the animal from which the sera was harvested (to reduce the potential for affinity purification of antibodies directed against any purification contaminants). These affinity-purified antibodies were then tested by immunoblotting to determine if they were capable of recognising each of the above purified proteins. Antibodies raised against the full length protein were capable of recognising all three purified proteins, consistent with the presence of antibodies directed against each domain. Antibodies raised against the SH3 domain were likewise only capable of recognising this domain. As expected, this antibody recognised only proteins including the GRAM PH or GAP domain. No signals were detected when antibodies were used that had been previously depleted by incubation with an excess of immobilised immunising proteins. These antibodies were then tested for their abilities to recognise endogenous GRAM from rat brain lysates by immunoblotting. All antibodies recognised one or more bands around 94 kDa. Since the antibodies raised against the PH+GAP domains consistently recognised three closely related bands of ˜94 kDa (and no other bands) from brain lysate, this antibody was used in most subsequent immunoblotting analyses. This antibody differentially recognises one or more of these bands in lysates from HeLa (human cervical carcinoma), SH-SY5Y (human neuroblastoma), and K562.

### Antibodies and DNA Constructs

Polyclonal antisera against GRAF1 were generated by immunising rabbits (RA-83/Ab1), (RA-84/Ab2), and a chicken (CH-9798/Ab4, used for immunofluorescence analysis) with recombinantly expressed human GRAM proteins. Purchased antibodies were: mouse anti-myc clone 9E10, mouse anti-tubulin (Sigma-Aldrich), rabbit anti-myc (Cell-Signalling Technology), mouse anti-dynamin, mouse anti-GIT1 (BD Transduction Laboratories), rabbit anti-synaptojanin Ra59 (Praefcke et al., 2004), mouse anti-paxillin, mouse anti-vinculin, rabbit anti-FAK (Abcam), mouse anti-pFAK (Biosource) and mouse anti-haemagglutinin (HA) clone 12CA5 (ROCHE Applied Science). All secondary antibodies and streptavidins were conjugated to Alexa Fluor 488, 546 or 647 (Molecular Probes). cDNA constructs encoding human GRAF1 (amino acids 1-759), GRAF1-BARPH (amino acids 1-383), GRAF1-SH3 (694-759) were amplified from IMAGE clone 30343863 using PCR and cloned into the pGEX-4T-2 vector (Amersham Biosciences) for bacterial expression and pCMVmyc vector with added Not1 site (JGW Anderson) or EGFP-C3 (Clontech) for mammalian expression. Amino acid substitutions K131E, K132E and R412D were created using PCR directed mutagenesis (Stratagene). Y-27632 was obtained. from Sigma. Praefcke, G. J., Ford, M. G., Schmid, E. M., Olesen, L. E., Gallop, J. L., Peak-Chew, S. Y., Vallis, Y., Babu, M. M., Mills, I. G., and McMahon, H. T. (2004). Evolving nature of the AP2 alpha-appendage hub during clathrin-coated vesicle endocytosis. EMBO J. 23, 4371-4383.

### Example 3

### Graf1-Dependent Endocytosis is Necessary for Cell Migration

### Overview:

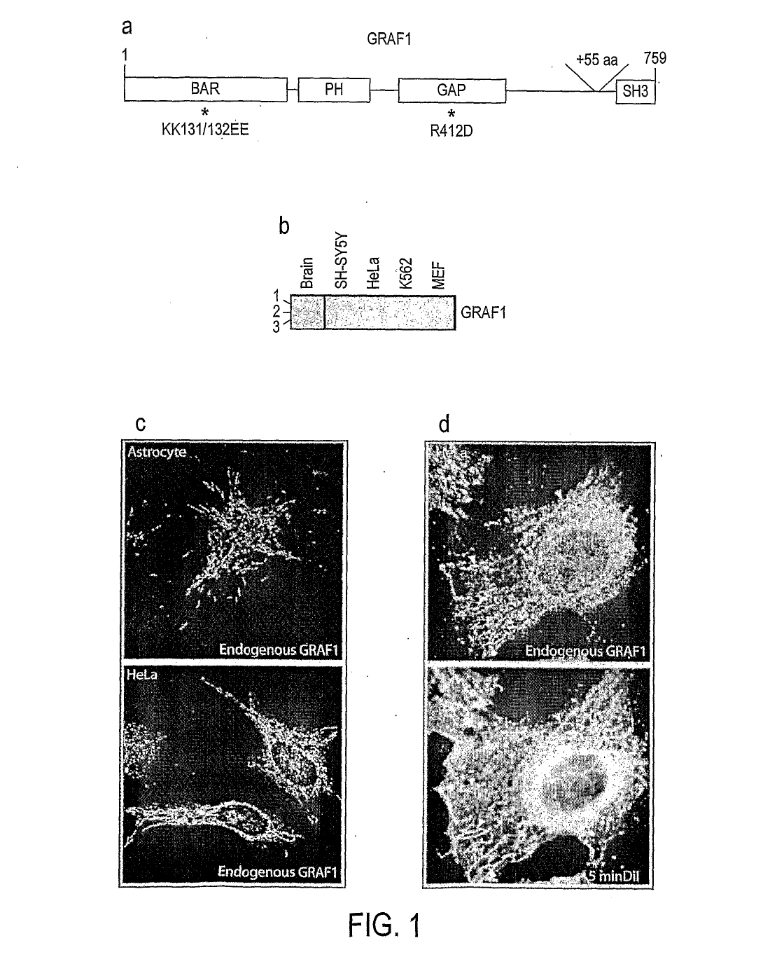

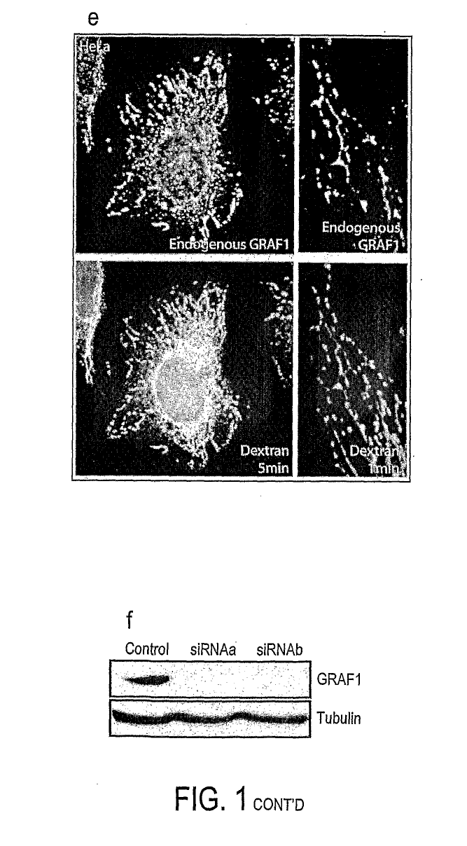

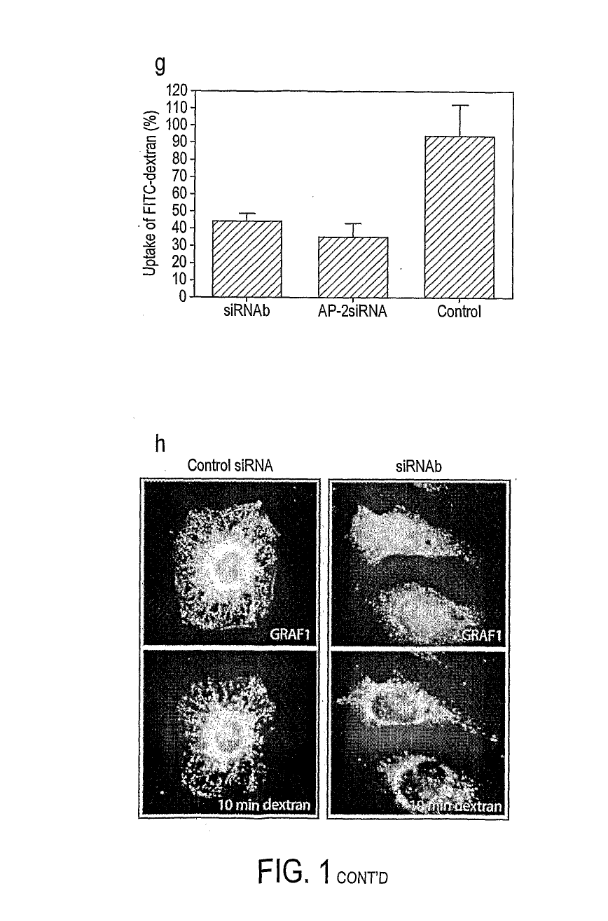

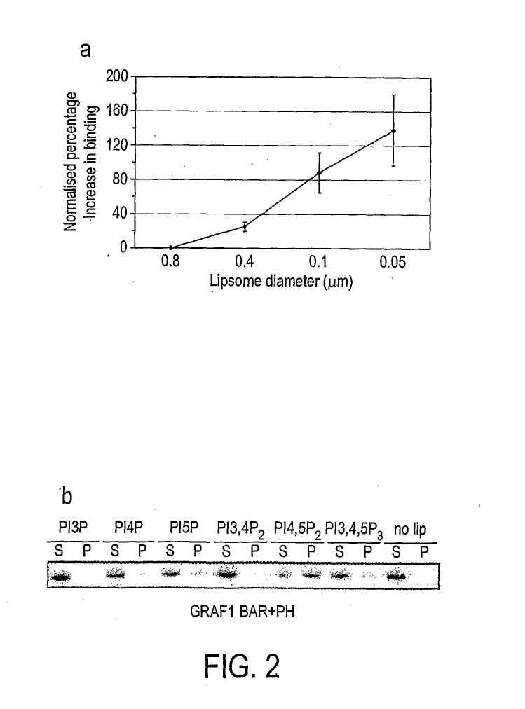

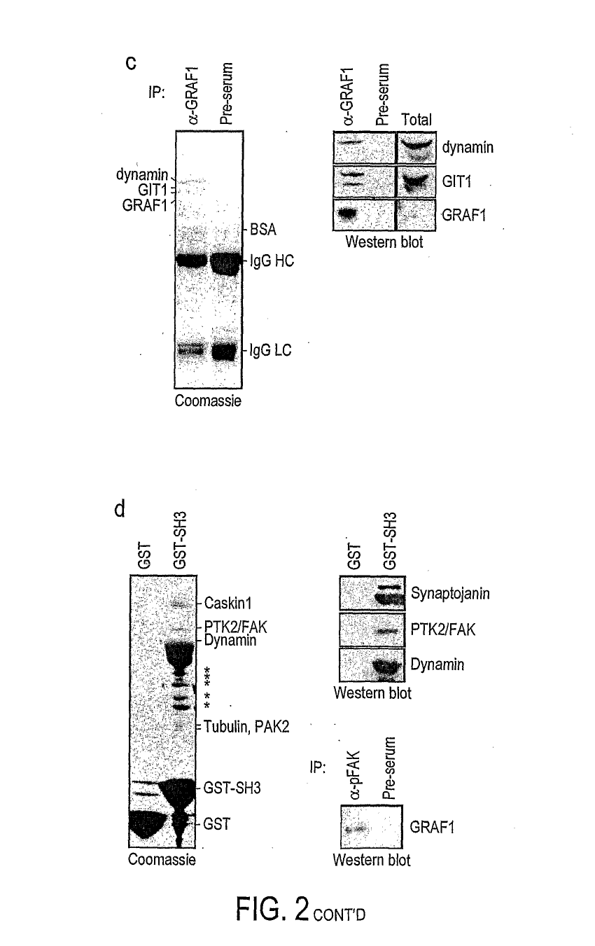

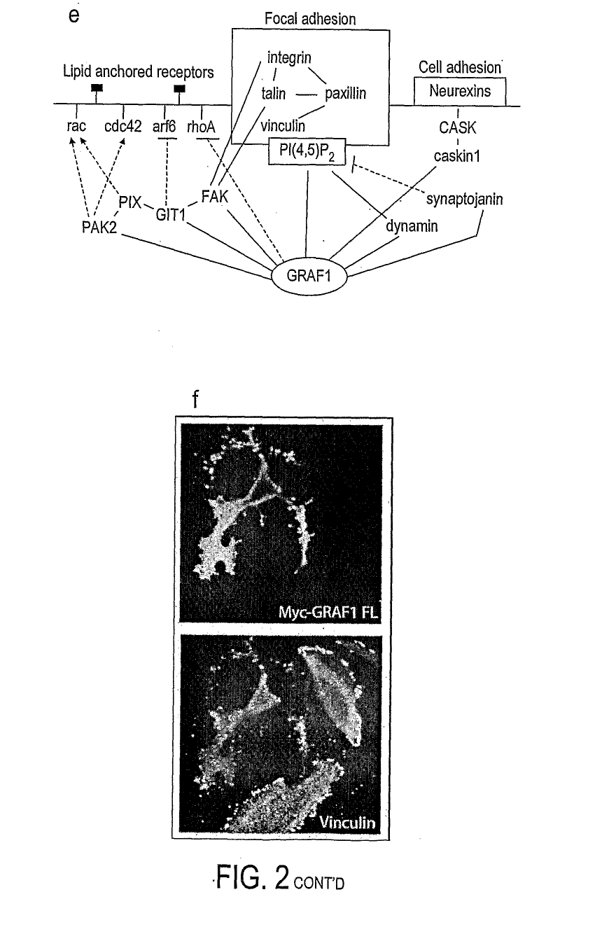

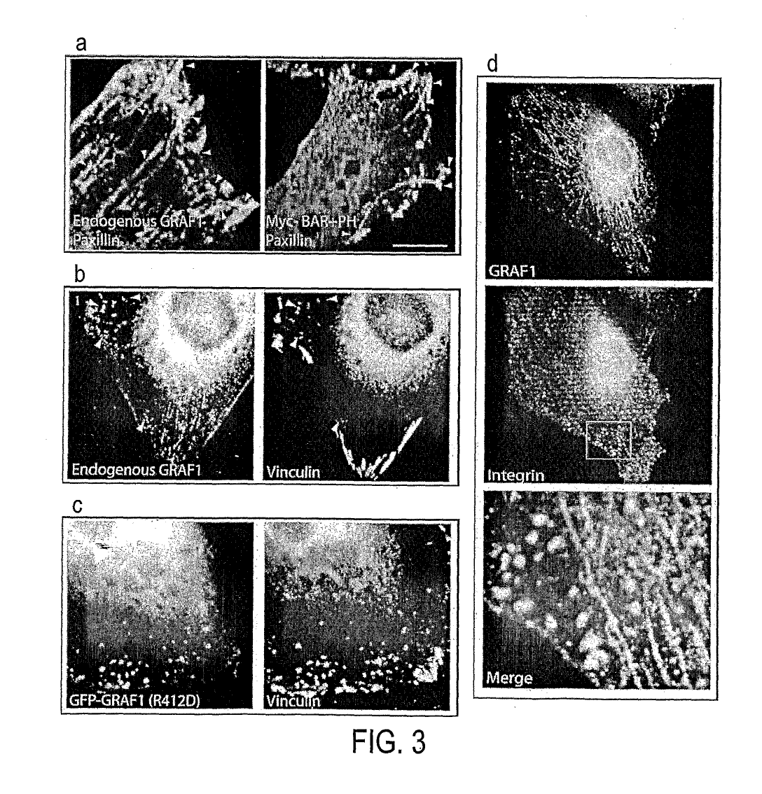

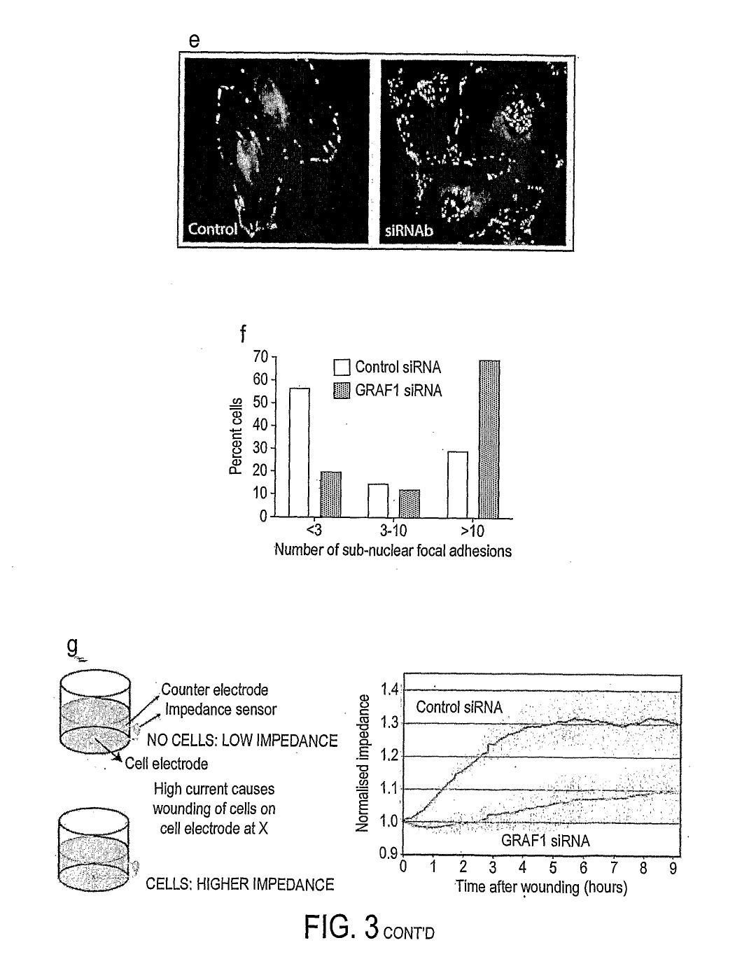

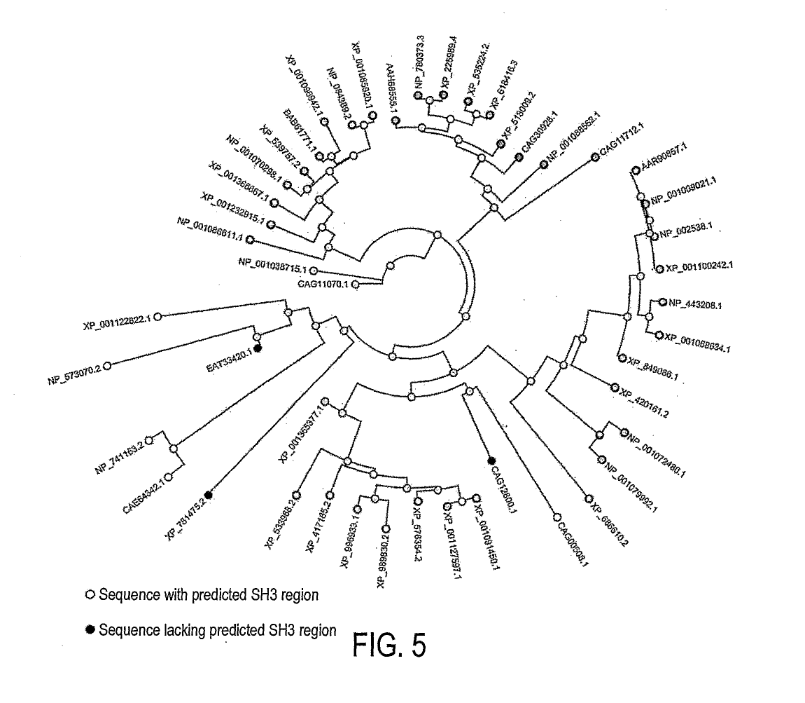

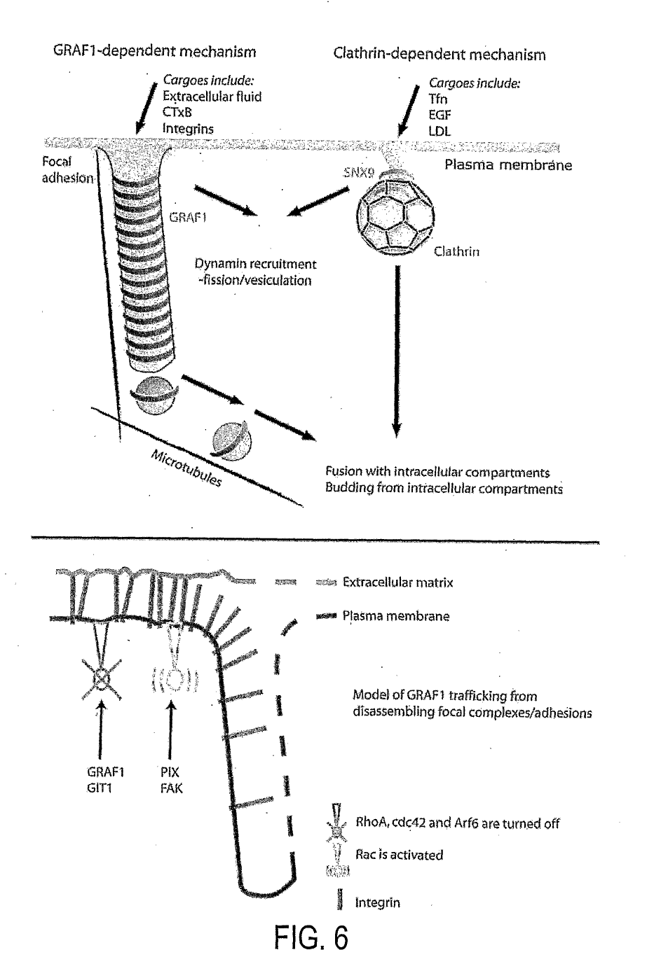

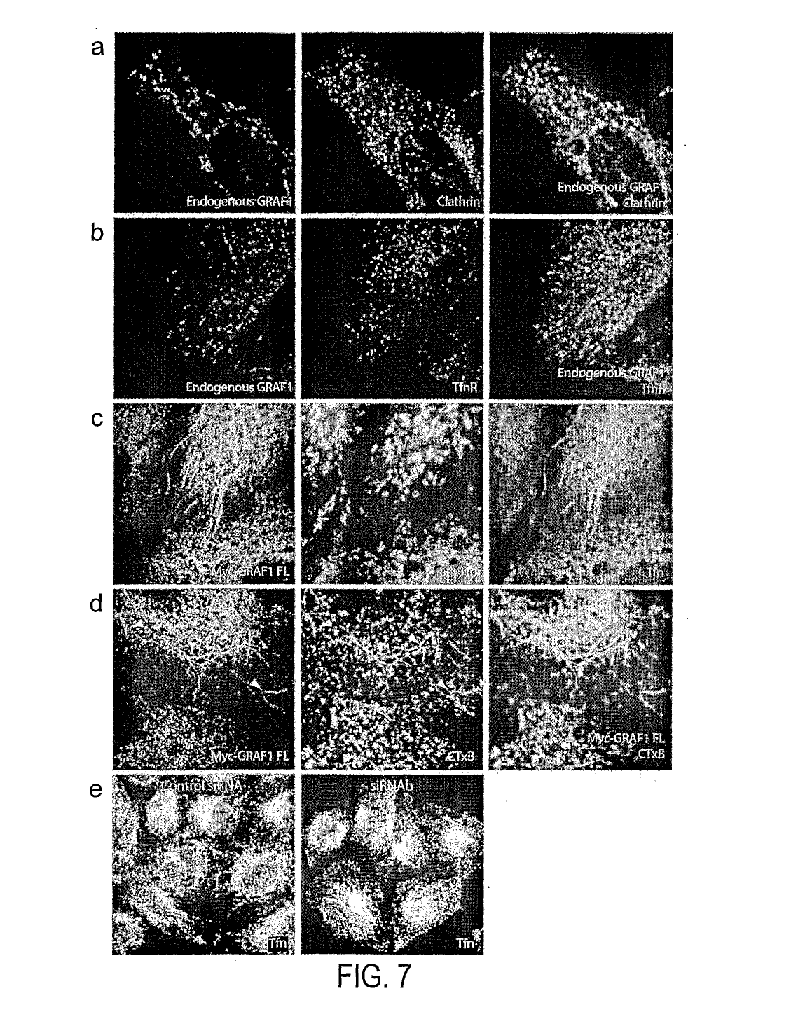

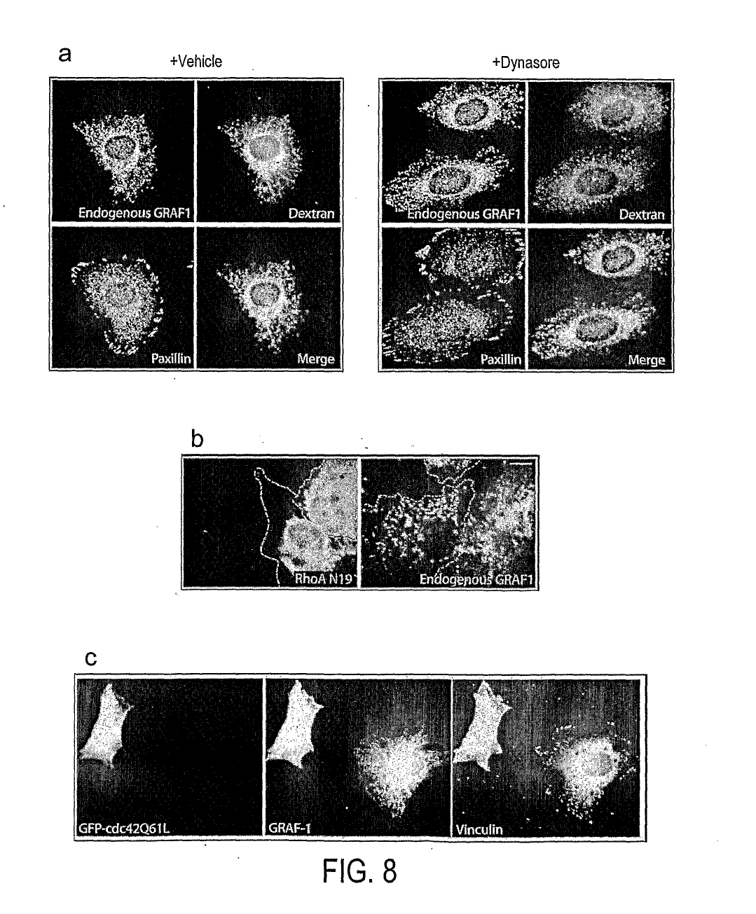

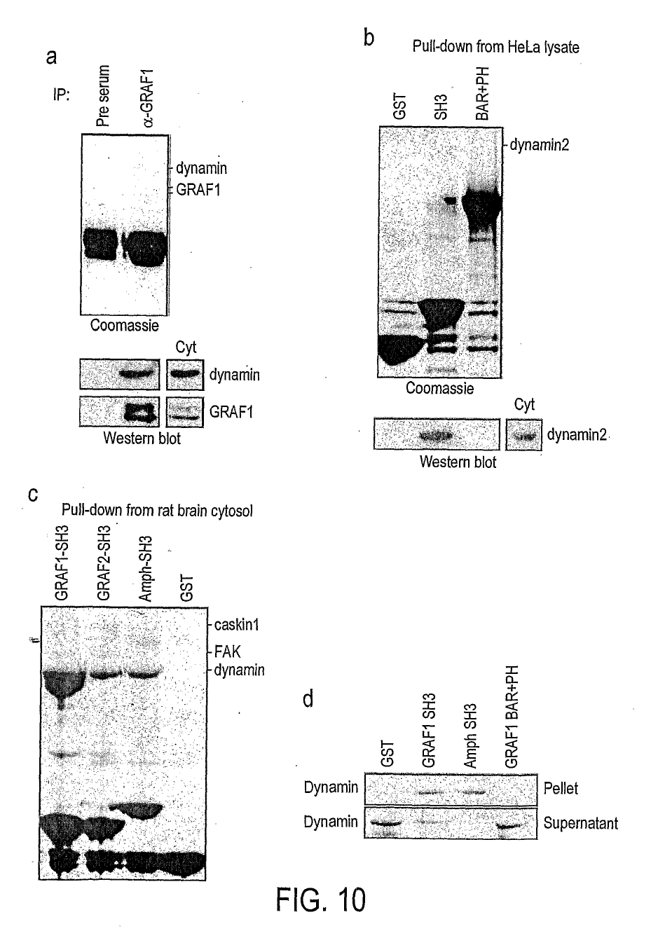

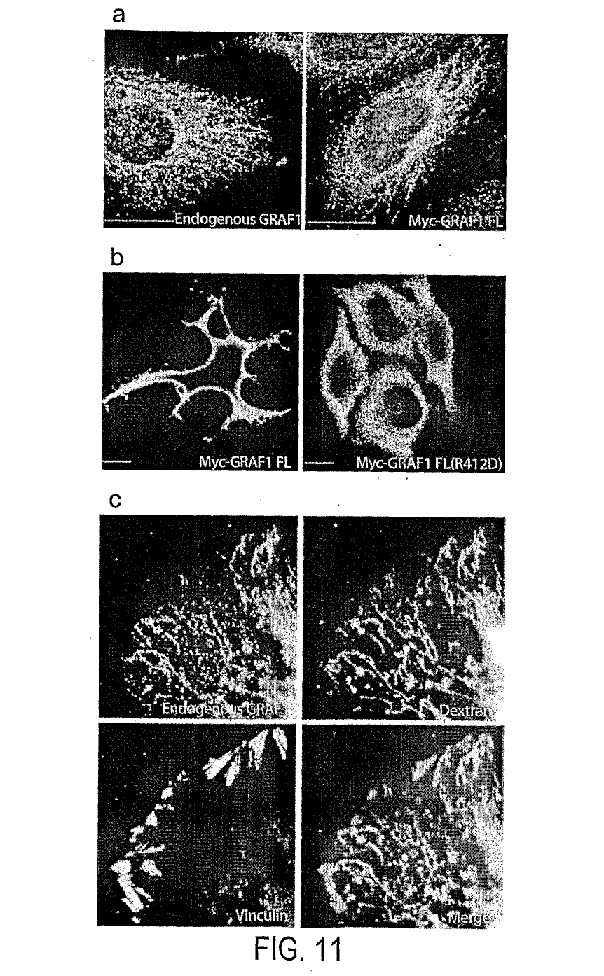

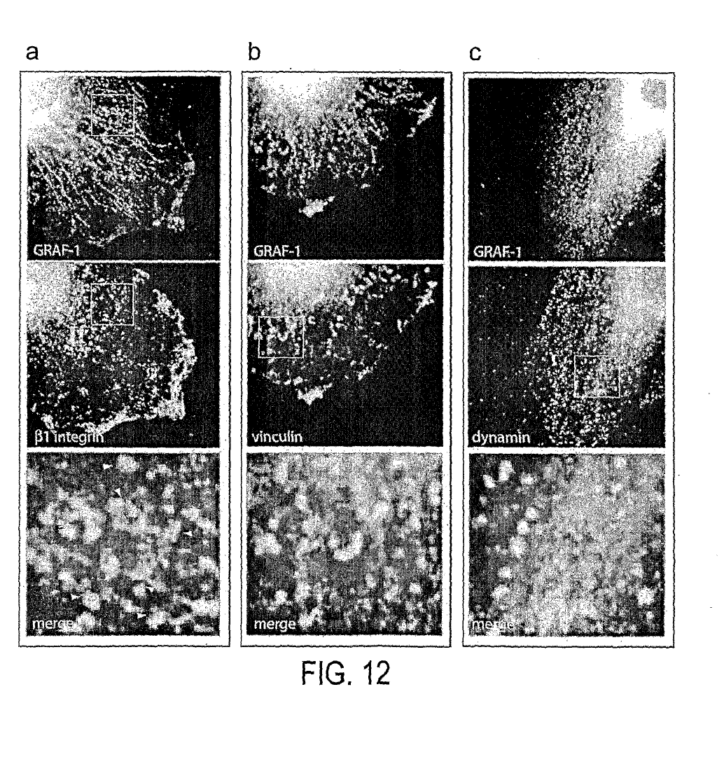

Cell migration requires the intricate coordination of membrane and protein redistribution, cytoskeletal changes, and focal complex/adhesion turnover1. The mechanisms by which this coordination occurs are unclear. The poorly understood promigratory phenotypes acquired by invading and metastasising cancer cells2 underline the importance of elucidating the cell biological mechanisms of migration. Members of the Rho family of small G-proteins have been shown to be master regulators of cell migration3. Here we show that the Rho GAP domain-containing protein GRAF1 regulates a major clathrin-independent endocytic pathway which is necessary for cell migration. GRAF1 localises to PtdIns(4,5)P2-enriched, tubular and punctate lipid structures in vivo via BAR and PH domains. We show that GRAM binds dynamin, GIT1, FAK, and PAK2. Since these proteins promote the disassembly of focal adhesions, which occurs in an endocytic manner4, 5, this places GRAF1 in a position to coordinate cell migratory events. We show that GRAF1 is necessary for turnover of focal complexes/adhesions and that GRAM-dependent endocytosis occurs from these sites in a small G-protein-dependent manner. GRAF1-dependent endocytosis therefore provides a novel cellular mechanism for the direct coupling of endocytosis with changes in cellular morphology necessary for cell migration. GTPase Regulator Associated with Focal Adhesion Kinase-1 (GRAF1) is a member of the diverse Rho GTPase activating protein (GAP) family. The GAP domain of GRAF1 exhibits GAP activity for RhoA and Cdc42 in vitro and favours the downregulation of RhoA activity in vivo 6, 7. GRAF1 is a brain-enriched protein containing PH, GAP and SH3 domains, and has an N-terminal region with homology to BAR domains (FIG. 1a). It is also expressed in primary fibroblasts and a variety of cell lines, including neuroblastoma and fibroblast cells (FIG. 1b). We found that GRAF1 was localised predominantly to tubular and punctate structures in astrocytes and HeLa cells by immunofluorescence (FIG. 1c). Since GRAF1 tubules were observed to frequently contact the periphery of these cells, suggestive of a role for GRAF1 in plasma membrane trafficking, we monitored endocytosis with either DU (to identify plasma membrane derived structures), or the fluid phase marker dextran (to highlight the lumen of endocytic structures), before staining for GRAF1. GRAF1 tubular structures extensively colocalised with both of these markers after 5 minutes, and even after 1 minute of incubation, indicative of an endocytic role for these tubules (FIG. 1d,e). Clathrin polymers should not be geometrically capable of stabilising tubular membranes and is not found on tubular membranes by electron microscopy. Indeed GRAF1-positive tubules were devoid of clathrin, and did not colocalise with the transferrin receptor or with internalised transferrin, consistent with a role in clathrin-independent endocytosis (FIG. 7a-c). Also, GRAF1-positive tubules accumulated Cholera Toxin Subunit B (CTxB), a marker used for the study of clathrin-independent endocytic pathways8 (FIG. 7d). To determine if GRAF1 was necessary for endocytosis via these tubules we depleted GRAF1 levels using siRNA. This treatment was capable of reducing GRAF1 levels to background as assessed by Western blots on tissue lysates and immunofluorescence but had no effect on transferrin endocytosis (FIG. 1f, g and FIG. 7e). GRAF1-depleted cells were then assessed for their ability to endocytose dextran, both by immunofluorescence and by a quantitative fluorimetric assay (FIG. 1g, h). GRAF1 depletion resulted in a 50-60% reduction of dextran endocytosis, similar to that observed by AP2 depletion (FIG. 1g), suggesting that GRAF1-mediated endocytosis and clathrin-mediated endocytosis account for roughly equal amounts of volume internalisation in these cells. Cells depleted of both AP2 and GRAF1 were not viable. Furthermore, GRAF1 depletion resulted in a large reduction in the amount of tubular endocytosis of dextran, suggesting that GRAF1 regulates the formation of these structures. To determine if this tubular endocytosis was dynamin-dependent we incubated cells with dynasore, a cell permeable inhibitor of dynamin function9, and analysed dextran uptake in these cells. Dynasore treated cells had a profoundly reduced ability to endocytose dextran in tubular structures, and GRAF1 was redistributed in these cells from tubular structures to basal punctae and rings. Small G-protein balance was also found to be important for this process, since overexpression of dominant ᵢ©active/− negative small G-proteins abolished the tubular morphology of endogenous GRAF1 (FIG. 8). To examine the role of the predicted N-terminal BAR domain of GRAF1 we incubated purified GRAF1 BAR+PH protein with liposomes of varying diameter and examined lipid binding by a co-sedimentation assay (FIG. 2a and FIG. 9a). GRAF1 BAR+PH was found to bind best to liposomes of small diameter, consistent with the presence of a membrane curvature-sensing BAR domain. Indeed, this protein was also capable of binding to tubular structures in vivo, its localisation being dependent on key lysine residues which are necessary for lipid binding in other BAR domain proteins 10; 11. The protein was also capable of generating tubules in vitro from spherical liposomes as examined by electron microscopy (FIGS. 9b and c). Using similar co-sedimentation assays with liposomes of varying phosphoinositide enrichments, we found that GRAF1 BAR+PH bound best to PtdIns(4,5)P2-enriched membranes (FIG. 2b). PtdIns(4,5)P2 is a plasma membrane-enriched phosphoinositide, and this finding is consistent with our observations on GRAF1-dependent endocytosis. Furthermore, PtdIns(4,5)P2 is found enriched in focal adhesion membranes and at the leading edge of migratory cells, where it has a complex life cycle controlling responses to migratory stimuli12. Tubular membranes might be expected to be stabilised by cytoskeletal elements in vivo, which may also provide directionality for the delivery of cargo to intracellular targets. Indeed treatment of GRAF1 BAR+PH overexpressing cells with the microtubule depolymerising agent nocodazole abolishes the tubular localisation of this overexpressed protein and during recovery colocalisation of GRAF1 and tubulin is observed (FIG. 9d). Using real time fluorescence microscopy we determined that these tubules are highly dynamic structures capable of extension, retraction, and high speed membrane trafficking (FIG. 9e and Movies). Taken together, these results highly suggest that the BAR domain of GRAF1 localises the protein to tubules in vivo and that it may play a role in the generation and stabilisation of these highly curved membranes, the directionality and extent of which is microtubule dependent. To determine interacting partners for GRAF1 we performed coimmunoprecipation experiments from rat brain cytosol with antibodies specific to GRAF1. GRAF1 was found in a complex with the membrane scission protein dynamin, as well as the Arf6 GAP protein GIT1 (FIG. 2c ᵢ® determined by mass spectrometry of Coomassie-stained bands and confirmed by Western blot). To identify other putative binding partners we performed pull down experiments with the SH3 domain of GRAF1 from rat brain cytosol (FIG. 2d). We found that the major interacting partner of this domain in this setting was dynamin, which we determined was direct by performing in vitro pull down experiments with purified dynamin. We verified that GRAF1 binds dynamin2 using co-immunoprecipitation and pull down experiments in HeLa cell lysates and that an antibody directed against a different region of GRAF1 co-immunoprecipitated with dynamin (FIG. 10). Our pull down experiments also identified FAK, PAK2, synaptojanin and caskin1 as interacting partners for GRAF1. FAK has previously been shown to interact with GRAF1 directly in vitro6. By immunoprecipitation with an antibody specific for pFAK (the active pY397 form of the protein present at focal adhesions13) we found that GRAF1 interacted with this form of the kinase (FIG. 2d). With further pull down experiments we showed that GRAF2 also binds FAK and caskin 1, suggesting that this homologous protein might play a similar role in different cell types (FIG. 10c). The novel interactions that we have determined, and other known interactions from the Human Protein Reference Database, are depicted together as an interactome in FIG. 2e. PAK, PIX, GIT1, and pFAK have been heavily implicated in the promotion of focal complex/adhesion disassembly4, 5 and a GIT1/PIX complex has been suggested to regulate trafficking between the plasma membrane and endosomes14. Furthermore, dynamin and microtubules have recently been shown to be involved in focal adhesion disassembly, which occurs in an endocytic manner5. To determine if GRAF1 also positively promoted focal adhesion/complex disassembly we overexpressed the full length protein in HeLa cells. While the distribution of overexpressed GRAF1 at low levels mimics that of the endogenous protein, higher expression levels result in a dose and time-dependent change in cellular morphology, with extensive membrane protrusions and arborisations that is dependent on the activity of the GAP domain. It also results in a dose-dependent reduction of focal adhesion markers such as vinculin and paxillin (FIG. 2f, FIGS. 11a and b). To further investigate the link between GRAF1 and focal complexes/adhesions we co-stained HeLa cells for endogenous, GRAF1 and focal complex/adhesion markers. We found that the peripheral ends of GRAF1-positive tubules are at sites of focal adhesion, consistent with a role in trafficking from these sites (FIG. 3a). Indeed, endocytic tubules accumulating dextran were also found to arise from these sites (FIG. 11c). In addition to a tubular localisation in fibroblasts, GRAF1 is also found on punctate structures throughout the cell. Many of these punctae are basically located and we found that these colocalise with focal complexes (FIG. 3b). Mutation of the arginine finger in the GAP domain of GRAF1 substantially increases the total amount of such colocalisation (FIG. 3c) showing that the GAP domain is ordinarily active and that this mutation induces a block in GRAF1-dependent processes that is usually overcome by changes in local small G-protein balance. If GRAF1-mediated trafficking positively regulates focal adhesion disassembly, we predicted that stimulation of focal adhesion disassembly should acutely increase the number of GRAF1-positive tubules and colocalisation with focal complex/adhesion markers. Treatment of HeLa cells with a Rho kinase inhibitor (which induces complex/adhesion disassembly) dramatically increased the numbers of GRAF1-positive tubules and increased the colocalisation of GRAF1 with 1-integrin, vinculin and dynamin (FIG. 3d and FIG. 12). Taken together, these data confirm our biochemical predictions of the action of the protein, and show that GRAF1-dependent endocytosis occurs from sites of disassembling focal complexes/adhesions. To determine if GRAF1-dependent endocytosis was itself necessary for focal adhesion disassembly, we depleted cells of GRAF1 and examined the number of focal adhesions in these cells (FIGS. 3e and f). GRAF1 depleted cells had a large increase in the number of focal adhesions, particularly in the subnuclear regions of these cells. We next examined the ability of these cells to migrate into an electronically induced wound, using quantitative measurements of impedance values (an increase in impedance reflecting migration of cells into the wounded area ᵢ© FIG. 3g). GRAF1-depleted cells were profoundly deficient in their ability to migrate. These data show that GRAF1-dependent endocytosis is necessary for focal adhesion disassembly and cell migration. Clathrin-independent endocytic pathways used by bacterial toxins, viruses and GPI-linked proteins have been well-studied15-17 but such work has been impeded by the lack of specific endogenous markers of the trafficking machinery required for these pathways. Caveolin1 and flotillin1 have been shown to be necessary for clathrin independent endocytic processes but the mechanisms by which they function remain unclear18, 19. Characterisation of the mechanisms underlying these pathways has had to heavily rely on their dependence on specific small G-proteins, and on their necessary upstream lipid domain organisation. Clustering of both specific cargo and small G proteins in lipid raft-like domains appears to support endocytosis. Interestingly, focal adhesions are domains with a high degree of membrane organisation and loss of integrin adhesion has been shown to promote endocytic trafficking20. Our work provides an endogenous marker for one such pathway that is dependent upon dynamin and small G proteins, and further work will address the nature of the specific endogenous cargoes that are endocytosed via this pathway and determine its precise relationship to clathrin independent tubules that have been observed to accumulate specific cargoes by electron microscopy techniques15. Our studies also provide further evidence for anatomical sites within the cell from which endocytosis may preferentially occur. We show that a single protein is capable of coordinating endocytosis and focal adhesion turnover, which is required for cell migration. Our data support a role for a GRAF1/GIT1/FAK/Dynamin complex in the downregulation of focal adhesions via the regulation of small G-proteins and tubular trafficking (FIG. 6). Deficiencies of membrane trafficking pathways are already extensively linked to human disease21. Interestingly, both the GIT1/PIX complex and a close GRAF1 homologue, Oligophrenin1, have been shown to be essential for the morphogenesis of dendritic spines22 and Oligophrenin 1 is often found mutated in patients with syndromic X-linked mental retardation23, where deficiency of a similar trafficking pathway is likely to be the primary cause of disease. GRAF1 is a putative tumour suppressor protein in haematopoietic cells24, 25, where deficiency of a similar pathway may contribute to pathogenicity. Hyperactivity of this pathway may also contribute to cancer cell invasion in solid organ malignancies. Indeed, overexpression or heightened activity of FAK is found in a wide variety of these cancers, and is currently studied as a putative cancer target26. Our results may contribute to providing specificity to the rational design of similar drug discovery programmes. We have shown how a single protein sits at the juncture between membrane trafficking and a cell physiological process. We predict that careful study of each member of the BAR domain-containing protein family will reveal roles in similar events whereby membrane trafficking and their other effector functions are specifically coordinated at defined sites within the cell.

### Materials and Methods

Protein expression and purification Antibodies and constructs used are described in supplemental methods. Recombinant proteins were expressed in a BL21 (DE3) pLysS E-coli strain as Glutathione Stransferase (GST)-fusion proteins and purified using glutathione-Sepharose 4B beads (Amersham Biosciences) and gel filtration on a sephacryl S-200 column (Amersham) as previously described27. For analysis of endogenous protein expression, cell lines were grown according to instructions from American Tissue Culture Collection, harvested and lysed in 1% NP-40 in PBS supplemented with protease inhibitors. After a 20,000 g centrifugation the supernatant was analysed by SDS-PAGE and immunoblotting. Protein and lipid interaction assays Liposomes from total brain lipids (FOLCH fraction I) (Sigma Aldrich), from synthetic lipids (Avanti Polar Lipids), and liposomes of a specified diameter were generated as previously described10. Liposome binding assays for lipid specificity and curvature sensitivity was performed as previously described10. Briefly, proteins were incubated together with liposomes followed by centrifugation and analysis of the pellet and supernatant by SDS-PAGE and Coomassie staining. For immunoprecipitation experiments, rat brain cytosol was generated by homogenisation of rat brains in buffer (25 mM HEPES, 150 mM NaCl, 1 mM DTT, 0.1% Triton X-100 and protease inhibitors), before centrifugation at 50,000 rpm for 30 mins at 4ᵢ [symbol] C. The supernatant was removed and added to protein A Sephorose 4B beads (Amersham Biosciensis) to which antibodies had been previously bound and incubated at 4° C. for 3 hrs. Beads were washed three times in buffer (25 mM HEPES, 150 mM NaCl) supplemented with 1% NP-40, and once in buffer without NP-40 before analysis by SDS-PAGE combined with immunoblotting or Coomassie staining. Pull-down experiments against rat brain cytosol using purified proteins and identification by mass-spectrometry were performed as previously described27. In vitro liposome tubulation assays were performed and analysed as previously described 10. Cell culture and transfections HeLa cells were grown in RPM1 1640 media (GIBCO) supplemented with L-Glutamine and 10% fetal bovine serum and transfected using Genejuice (Novagen) for transient protein expression. For primary cultures, rat hippocampal neurons/astrocytes were prepared by trypsin digestion and mechanical trituration from E18 or P1 Sprague-Dawley rats and plated onto poly L-lysine coated coverslips. Cells were cultured in B27-supplemented Neurobasal media. For GRAF1 depletion, HeLa cells were transfected with stealth siRNAs specific against human GRAF1 (Invitrogen) using Lipofectamine 2000 (Invitrogen) according to manufacturers instructions. Cells were cultured for an additional 48 hrs for efficient silencing of the GRAF1 expression. Stealth Block-it siRNA (Invitrogen) was used as a control. AP2 siRNA was used as previously described28. Trafficking assays For immunofluorescent trafficking assays, biotinylated holo-transferrin, (Sigma Aldrich), Alexa Fluor 546-conjugated CTxB, DiI, FITC-dextran (10 kDa MW, used for fluorimetric uptake assay), and biotinylated dextran (10 kDa MW, used for immunofluorescent uptake assays) (Molecular Probes), were diluted in pre-warmed media, added to cells and incubated for time periods and temperatures as described in figure legends. After washing, cells were fixed and subjected to immunofluorescent analysis as described below. For quantitative analysis of dextran endocytosis, HeLa cells in 35 mm dishes were transfected with siRNAs/control siRNAs 48 hrs prior to the experiment. Fluorescein isothiocyanate (FITC)-dextran (Sigma-Aldrich) was diluted in media to a concentration of 1 mg/ml and added to cells before incubation for 15 min at the indicated temperature. Cells were washed twice in media and once in PBS before lysis in 1% NP-40 in PBS supplemented with protease inhibitors. The lysate was centrifuged at 20,000 g for 20 min at 40° C. and the protein concentration in the supernatant was measured using the BCA Protein Assay Kit (Pierce) for normalisation. The amount of FITC-dextran in the supernatant was measured, as the emission at 515 nm after exciting at 488 nm using a FP-6500 spectrofluorometer with Spectra Manager software (JASCO). Imaging For immunofluorescent analysis, HeLa cells were fixed in 3% paraformaldehyde in phosphate-buffered saline (PBS) for 15 min at 37ᵢ [symbol] C (to preserve intracellular tubules which are disrupted by fixation at lower temperatures), washed and blocked in 5% goat serum, with 0.1% saponin, in PBS before staining with the appropriate antibodies in 1% goat serum, 0.1% saponin in PBS using standard protocols. Confocal images were taken sequentially using a BioRad Radiance system and LaserSharp software (BioRad). Epifluorescence images were taken using a Zeiss Axioimager Z1 system with AxioVision software. For real time microscopy, transfected cells on glass-bottom Petri dishes (WillCo Wells BV, Amsterdam) were washed with buffer (125 mM NaCl, 5 mM KCl, 10 mM D-glucose, 1 mM MgCl2, 2 mM CaCl2 and 25 mM HEPES) and images were taken using a 5-live scanning microscope (Zeiss) or spinning disc confocal system (Improvision) with subsequent analysis in LSM Image Browser (Zeiss), ImageJ or Volocity (Improvision). Biophysical cell recordings 105 HeLa cells were transfected with siRNA against GRAF1 or control siRNA for 24 hrs before plating into chambers of 8W1E electrode arrays (Applied Biophysics) and incubated at 37° C. with 5% CO2. Impedance values between the electrode and counter electrode were recorded continuously at a 15 kHz oscillator frequency from each array using an ECIS 1600 system with elevated field module (Electronic Cell-substrate Impendence Sensing, Applied Biophysics). Cell attachment, spreading and layer confluence were verified electrically and microscopically before electrical wounding at 45 kHz, 4V, for 10 s with subsequent recording from electrodes using the same parameters as pre-wounding. Data was normalised to initial electrode impedance value for each wounding experiment.

### Example 4

### The Role of Graft in Endocytosis and Cell Migration

Cell migration requires the coordination of membrane and protein redistribution, cytoskeletal changes, and focal complex/adhesion turnover. The mechanisms by which this coordination occurs have been unclear in the prior art. Rho family small G-proteins have been shown to be master regulators of cell migration. Here we show that a Rho GAP domain-containing protein, GRAF1, regulates a major clathrin-independent endocytic pathway responsible for the internalisation of bacterial exotoxins, GPI-linked proteins, and extracellular fluid. We show that GRAF1 localises to PtdIns(4,5)P-2-enriched tubular and punctate lipid structures in vivo via its N-terminal BAR and PH domains, and that GRAF1 binds dynamin, GIT1, FAK, and PAK2. These latter proteins promote the disassembly of focal adhesions, placing GRAF1 in a position whereby it may coordinate cell migratory events. We show that GRAF1 is necessary for turnover of focal complexes/adhesions, that GRAF1-dependent endocytosis occurs from these sites in a small G-protein dependent manner, and that GRAF1 is necessary for cell migration.

### Example 5

### Organismal Studies

Only one member of the GRAF family (D-GRAF for Drosophila GRAF) exists in Drosophila melanogaster. Since flies can be genetically-manipulated more readily than other species with a single GRAF family member, this organism was chosen as a model in which to study the physiological functions of GRAF family proteins. Two independent D. melanogaster transgenic lines have been produced that express D-GRAF-GFP at low and high levels under the control of the UAS promoter. These may be crossed with GAL4 lines to drive DGRAF-GFP expression in specific tissues during development and beyond to examine its localisation and the effect of overexpression. Purified D-GRAF-SH3 has been injected into rabbits in order to generate polyclonal antibodies to this protein which will be used to examine the endogenous tissue and subcellular distribution of GRAF1, and cell types with greatest expression will be focused on in transgenic experiments. Four independent RNAi lines for D-GRAF have also been received from collaborative sources; and expression of RNAi will be targeted to tissues in which D-GRAF is expressed in order to examine its role in these tissues during development and adulthood. A D-GRAF null fly will also be produced to study this by another, more stringent method. Any phenotypes found will be extensively investigated. Biochemical and cell biological interrogation of D-GRAF function, as has been performed for GRAF1 in this dissertation, will also be carried out.

### Mammalian Studies

While flies have one GRAF family orthologue, mice and humans have 4 members of this family. As a model for human biology and disease, the mouse (Mus musculus) is well established. Therefore, to understand better the function of GRAF1 proteins in mammals (and in the context of the other GRAF family members), an approach to create GRAF1-null mice is underway (in collaboration with Andrew McKenzie) and is close to completion. Embryonic stem cells carrying a GRAF1-null mutation have been produced and injected into mouse blastocysts which were then implanted into recipient mice. Chimaeric mice produced from these procedures are currently breeding. Resultant knock-out mice will be compared with heterozygous and wild-type littermates in order to identify phenotypes (if any) associated with GRAF1 loss. Initial viability of these mice might be tentatively predicted by the low embryonic levels of GRAF1 expression and the postnatal surge in expression in the brain. Tissues will be examined for structural defects, and whole mice examined for behavioural abnormalities. Any phenotypes associated with loss of GRAF1 expression will be extensively investigated. Since GRAF1 is required for cell migration, and since migratory cells in adult mice include astrocytes and leukocytes, these cells will be examined for abnormalities. In a whole animal setting, this will include examination of the abilities of knock-out mice to form glial scars, and eradicate infectious insults such as subcutaneous bacterial inocula. Knock-out mice will be investigated for their propensity to progress to leukaemic phenotypes and will be crossed with mice that are prone to Acute Myeloid Leukaemia-like malignancies to examine if there exists any enhancement of the progression of this disease in a GRAF1-null background. Primary cells will be isolated from these animals and tested in in vitro endocytic and cell migration assays.

### Summary

The results presented herein have shown that the tumour suppressor protein GRAF1 lines an extensive system of tubular endocytic membranes that are independent of Clathrin, Caveolin1 and Flotillin1. GRAF1 is necessary for endocytosis into these membranes and its N-terminal portion (which comprises functional BAR and PH domains) acts to stabilise their high curvatures. These membranes are responsible for about half of fluid phase uptake in fibroblastic cells, are derived from the plasma membrane predominantly at the cell periphery, and are necessary for the delivery of cargo to the Golgi apparatus. Cargoes for this pathway include bacterial exotoxins and GPI-linked proteins. GRAF1-dependent endocytosis occurs preferentially from adhesion sites. The C-terminal portion of GRAF1 is necessary for its function and comprises an active RhoGAP domain, and SH3 domain which interacts with a variety of proteins involved in focal adhesion disassembly. GRAF1 is necessary for focal adhesion disassembly and cell migration to proceed. These studies suggest a novel mechanism for focal adhesion disassembly and cell migration that occurs through GRAF1-dependent endocytosis of cell-matrix adhesion proteins and/or associated microdomain-associated lipids. GRAF1-related proteins appear to function in similar manners. These results provide a framework for the understanding of human disease processes such as mental retardation and malignancy to which aberrant expression of GRAF1 and related proteins are linked. Future research will use model organisms to further explore the normal physiological functions of GRAF1, and how these become dysregulated in disease.

### REFERENCES TO EXAMPLES SECTION

1. de Curtis, I. Cell migration: GAPs between membrane traffic and the cytoskeleton. EMBO Rep 2, 277-81 (2001).
2. Christofori, G. New signals from the invasive front. Nature 441, 444-50 (2006).
3. Raftopoulou, M. & Hall, A. Cell migration: Rho GTPases lead the way. Dev Biol 265, 23-32 (2004).
4. Zhao, Z. S., Manser, E., Loo, T. H. & Lim, L. Coupling of PAK-interacting exchange factor PIX to GIT1 promotes focal complex disassembly. Mol. Cell Biol. 20, 6354-63 (2000).
5. Ezratty, E. J., Partridge, M. A. & Gundersen, G. G. Microtubule-induced focal adhesion disassembly is mediated by dynamin and focal adhesion kinase. Nat. Cell Biol. 7, 581-90 (2005).
6. Hildebrand, J. D., Taylor, J. M. & Parsons, J. T. An SH3 domain-containing GTPase-activating protein for Rho and Cdc42 associates with focal adhesion kinase. Mol. Cell Biol. 16, 3169-78 (1996).
7. Taylor, J. M., Macklem, M. M. & Parsons, J. T. Cytoskeletal changes induced by GRAF, the GTPase regulator associated with focal adhesion kinase, are mediated by Rho. J. Cell Sci. 112 (Pt 2), 231-42 (1999).
8. Nichols, B. J. A distinct class of endosome mediates clathrin-independent endocytosis to the Golgi complex. Nat Cell Biol 4, 374-8 (2002).
9. Macia, E. et al. Dynasore, a cell-permeable inhibitor of dynamin. Dev Cell 10, 839-50 (2006).
10. Peter, B. J. et al. BAR domains as sensors of membrane curvature: the amphiphysin BAR structure. Science 303, 495-9 (2004).
11. Gallop, J. L. et al. Mechanism of endophilin N-BAR domain-mediated membrane curvature. EMBO J. 25, 2898-2910 (2006).
12. Ling, K., Schill, N. J., Wagoner, M. P., Sun, Y. & Anderson, R. A. Movin' on up: the role of PtdIns(4,5)P(2) in cell migration. Trends Cell Biol 16, 276-84 (2006).
13. Schaller, M. D. et al. pp 125FAK a structurally distinctive protein-tyrosine kinase associated with focal adhesions. Proc Natl Acad Sci USA 89, 5192-6 (1992).
14. Lahuna, O. et al. Thyrotropin receptor trafficking relies on the hScrib-betaPIXGIT1-ARF6 pathway. Embo J 24, 1364-74 (2005).
15. Kirkham, M. et al. Ultrastructural identification of uncoated caveolinindependent early endocytic vehicles. J. Cell Biol. 168, 465-76 (2005). °
16. Lamaze, C. et al. Interleukin 2 receptors and detergent-resistant membrane domains define a clathrin-independent endocytic pathway. Mol. Cell 7, 661-71 (2001).
17. Kirkham, M. & Parton, R. G. Clathrin-independent endocytosis: new insights into caveolae and non-caveolar lipid raft carriers. Biochim. Biophys. Acta 1746, 349-63 (2005).
18. Glebov, O. O., Bright, N. A. & Nichols, B. J. Flotillin-1 defines a clathrin independent endocytic pathway in mammalian cells. Nat Cell Biol 8, 46-54 (2006).
19. Hommelgaard, A. M. et al. Caveolae: stable membrane domains with a potential for internalization. Traffic 6, 720-4 (2005).
20. del Pozo, M. A. et al. Phospho-caveolin-1 mediates integrin-regulated membrane domain internalization. Nat Cell Biol 7, 901-8 (2005).
21. Howell, G. J., Holloway, Z. G., Cobbold, C., Monaco, A. P. & Ponnambalam, S. Cell biology of membrane trafficking in human disease. Int Rev Cytol 252, 1-69 (2006).
22. Govek, E. E. et dl. The X-linked mental retardation protein oligophrenin-1 is required for dendritic spine morphogenesis. Nat. Neurosci. 7, 364-72 (2004).
23. Zanni, G. et al. Oligophrenin 1 mutations frequently cause X-linked mental retardation with cerebellar hypoplasia. Neurology 65, 1364-9 (2005).
24. Borkhardt, A. et al. The human GRAF gene is fused to MLL in a unique t(5;11)(q31;q23) and both alleles are disrupted in three cases of myelodysplastic syndrome/acute myeloid leukemia with a deletion Sq. Proc. Natl. Acad. Sci. USA 97, 9168-73 (2000).
25. Bojesen, S. E. et al. Characterisation of the GRAF gene promoter and its methylation in patients with acute myeloid leukaemia and myelodysplastic syndrome. Br. J. Cancer 94, 323-32 (2006).
26. McLean, G. W. et al. The role of focal-adhesion kinase in cancer—a new therapeutic opportunity. Nat Rev Cancer 5, 505-15 (2005).
27. Praefcke, G. J. et al. Evolving nature of the AP2 alpha-appendage hub during clathrin-coated vesicle endocytosis. EMBO J. 23, 4371-83 (2004).
28. Motley, A., Bright, N. A., Seaman, M. N. & Robinson, M. S. Clathrin-mediated endocytosis in AP-2-depleted cells. J. Cell Biol. 162, 909-18 (2003).

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1. A method of identifying a modulator of clathrin-independent endocytosis, said method comprising
(i) providing a GRAF protein, said GRAF protein comprising a GAP domain;
(ii) providing a candidate modulator; and
(iii) determining an effect of said candidate modulator on GAP activity of said GRAF protein,
wherein a change in the GAP activity of said GRAF protein in the presence of said candidate modulator identifies said candidate modulator as a modulator of clathrin-independent endocytosis.

2. A method according to claim 1, said method comprising
(i) providing first and second samples of a GRAF protein, said GRAF protein comprising a GAP domain;
(ii) providing a candidate modulator;
(iii) contacting said second sample of GRAF protein with said candidate modulator;
(iv) determining the effect of said candidate modulator on the GAP activity of said GRAF protein by assaying the GAP activity of said first and second samples of GRAF protein;
wherein a difference in the GAP activity between said first and second samples of GRAF protein identifies said candidate modulator as a modulator of clathrin-independent endocytosis.

3. A method according to claim 2 wherein when the GAP activity is higher in said second sample than said first sample, the candidate modulator is identified as a stimulator or promoter of clathrin-independent endocytosis.

4. A method according to claim 2 wherein when the GAP activity is lower in said second sample than said first sample, the candidate modulator is identified as an inhibitor or suppressor of clathrin-independent endocytosis.

5. A method according to claim 1 wherein the GAP activity is assayed using RhoA as a substrate GTPase.

6. A method according to claim 1 wherein assaying the GAP activity comprises a tamra-GTP hydrolysis assay.

7. A method according to claim 1 wherein said GRAF protein comprises a polypeptide of at least 200 amino acid residues, and wherein said polypeptide comprises a GRAF GAP domain having at least 60% identity to the amino acid sequence 364-563 of human GRAF1.

8. A method according to claim 7 wherein said polypeptide comprises amino acid sequence corresponding to at least amino acids 364-563 of human GRAF1.

9. A method according to claim 1 further comprising performing an endocytic assay.

10. A method according to claim 1 further comprising performing an adhesion assay.

11. A method according to any preceding claim 1 further comprising performing a selectivity assay.

12. A method according to claim 1 further comprising assaying for modulators of FAK activity in vitro.

13. A method according to claim 1 further comprising assaying for modulators of GRAF RhoGAP activity in vitro.

14. A method according to claim 1 further comprising assaying for modulators of GRAF-FAK interaction in vitro.

15. A method according to claim 1 further comprising assaying for changes in GRAF distribution in cells.

16. A method according to claim 1 further comprising assaying for specific modulators of endocytic routes in vivo.

17. A method according to claim 1 further comprising comparing the GAP activity to a third sample GAP activity of a third sample of GRAF1 protein, said third sample comprising a mutant GRAF protein harbouring a mutation in its a GAP domain of said mutant GRAF protein corresponding to a R412D mutation of human GRAF1.

18. A method according to claim 1 further comprising manufacturing a quantity of the identified modulator of clathrin-independent endocytosis.

19-20. (canceled)

21. A composition comprising a GRAF polypeptide having a mutation at an amino acid residue corresponding to amino acid 412 of human GRAF1.

22. A composition according to claim 21 wherein said mutation is R412D.

23. (canceled)

24. A method for treating solid cell malignancy cancer or immuno suppressing a subject comprising: administering to the subject a modulator of clathrin-independent endocytosis.
